(19) United States
(12) Patent Application Publication
(10) Pub. No.: US 2025/0213738 A1
(43) Pub. Date: Jul. 3, 2025

(54) DETECTOR AND STERILIZATION SYSTEM

(71) Applicant: SHENZHEN MUXIN TECHNOLOGY CO., LTD., SHENZHEN, GUANGDONG (CN)

(72) Inventors: CHANG ZHAN, SHENZHEN (CN); XUANZHE WEI, SHENZHEN (CN); RONGJUN XIA, SHENZHEN (CN)

(21) Appl. No.: 19/062,642

(22) Filed: Feb. 25, 2025

Related U.S. Application Data
(63) Continuation-in-part of application No. 18/802,057, filed on Aug. 13, 2024, now Pat. No. 12,257,361.
Application No. 18/802,057 is a continuation of application No. PCT/CN2024/107519, filed on Jul. 25, 2024.

(30) Foreign Application Priority Data
Dec. 29, 2023 (CN) 202311862770.2
Aug. 20, 2024 (CN) 202422025991.0
Nov. 13, 2024 (CN) 202422777628.4

(51) Int. Cl. A61L 2/00; H05K 9/00

(52) U.S. Cl. CPC A61L 2/0029; H05K 9/0024; H05K 9/006

(57) ABSTRACT

A detector includes a housing assembly, a detection assembly and a shielding assembly. The detection assembly includes a first housing, a detection circuit board and a probe, the detection circuit board is disposed in the first housing and electrically connected with the probe, the detection circuit board includes a sensitive element, and a first end of the probe is fixed in the first housing while a second end stretches out of the first housing; the housing assembly includes a collision housing and a pressing portion that are capable of sliding in relative to each other, the detection assembly is located below a bottom of the pressing portion, the collision housing is used for abutting against a sampling part, and the pressing portion is used for driving the detection assembly to move towards the sampling part, so as to pierce the probe into the sampling part for detection.

### CROSS-REFERENCE TO RELATED APPLICATIONS

This application is a Continuation-in-Part of the U.S. application Ser. No. 18/802,057 filed on Aug. 13, 2024, and entitled “DETECTOR AND STERILIZATION SYSTEM” now pending, and the application claims also priority of Chinese Patent Application No. CN2024220259910, filed on Aug. 20, 2024, and Chinese Patent Application No.CN2024227776284, filed on Nov. 13, 2024, the entire contents of which are incorporated herein by reference.

### TECHNICAL FIELD

The present disclosure relates to the technical field of health detection, and in particular to a detector and a sterilization system.

### BACKGROUND

In a routine medical detection environment, when a to-be-detected object is subjected to subcutaneous sampling detection, the help of a detector is required. The detector usually includes a detection instrument for driving a guide needle to move, and when in detection, the guide needle and a probe are usually driven by the detector to pierce a sampling part, thus obtaining body indexes through a detection circuit board inside the detector. To guarantee the medical health, the detector must be sterilized before delivering or sampling, generally sterilized through electromagnetic waves or irradiation, and however the electromagnetic waves or irradiation may affect the detection circuit board in the detector, leading to a fault occurred to the detection circuit board during sterilization.

### SUMMARY

Embodiments of the present disclosure provide a detector and a sterilization system, to address or ease one or more technical issues in the prior art.

As one aspect of embodiments of the present disclosure, embodiments of the present disclosure provide a detector, and the detector includes a housing assembly, a detection assembly and a shielding assembly.

The detection assembly includes a first housing, a detection circuit board and a probe, wherein the detection circuit board is arranged in the first housing and is electrically connected with the probe; the detection circuit board includes a sensitive element; a first end of the probe is fixed in the first housing, and a second end extends out of the first housing.

The housing assembly includes a collision housing and a pressing portion that are capable of sliding relative to each other; the detection assembly is located below a bottom of the pressing portion; the collision housing is used for abutting against a sampling part; the pressing portion is used for driving the detection assembly to move towards the sampling part, so as to pierce the probe into the sampling part for detection.

When the detector is sterilized through an irradiation ray, the shielding assembly is used for blocking part of the irradiation ray to protect the sensitive element; the shielding assembly includes a side plate connected to the detection circuit board, and a top plate connected to the side plate; the side plate is adjacent to the sensitive element; and the top plate is located on one side of the sensitive element away from the detection circuit board.

As another aspect of embodiments of the present disclosure, embodiments of the present disclosure provide a sterilization system, the sterilization system includes a bearing frame, and the bearing frame configured to cooperate with an irradiation source for sterilization; the bearing frame is located on one side of the irradiation source; one side of the bearing frame that faces the irradiation source is provided with a plurality of bearing portions; each bearing portion is configured to place a detector; the detector includes a housing assembly, a detection assembly, and a shielding assembly; and when the detector is placed on each bearing portion, the bearing portion is configured to correct an orientation of the detector, so that the shielding assembly in the detector is located on a path that the irradiation source irradiates the detector.

Optionally, a center of the bearing frame is arranged correspondingly to the irradiation source; the bearing portions are bearing slots provided in the bearing frame; the bearing slot in the middle of the bearing frame is vertical, and other bearing slots adjacent to the middle bearing slot tilt towards one side of the irradiation source; and if the bearing slots are farther from the irradiation source, tilting angles of the bearing slots are larger.

Optionally, the sterilization system further includes at least one fastener of a magnetic suction fastener and a clip fastener; the fastener is configured to fix the detector on the bearing frame; the bearing portions are further provided with a plurality of first cooling holes; the sterilization system further includes a cover plate; the cover plate is configured to cover one side where the detector is located to fix the detector in cooperation with the bearing portions; the cover plate has a counterpoint slot for accommodating at least part of the detector, and a plurality of second cooling holes.

The bearing portions are further provided with counterpoint structures that are used in cooperation with the detector or another counterpoint structure on an outer package of the detector, so that the detector is capable of being arranged according to a preset orientation; and when the outer package of the detector has another counterpoint structure, the detector has a first counterpoint structure, and the outer package has a second counterpoint structure used in cooperation with the first counterpoint structure, so that the detector is arranged in the outer package according to the preset orientation.

When the embodiments of the present disclosure adopt the above-mentioned technical solution to irradiate the detector for sterilization, the irradiation ray is emitted to a sensitive element on the detection circuit board from one side of the first housing, and the shielding assembly can block the irradiation ray emitted to the sensitive element and form a total-shadow shielding zone not affected by the irradiation ray; and adjusting the position and size of the shielding assembly enables the total-shadow shielding zone formed by the shielding assembly to cover the sensitive element, and weakens or eliminates the irradiation influence on the sensitive element by the irradiation ray, thus relieving a situation that the detection circuit board cannot work normally due to a failure of the sensitive element affected by the irradiation. That is, the present disclosure can also protect the detection circuit board from being damaged in a case of guaranteeing the irradiation ray to sterilize the detector. At the same time, at least part of the total-shadow shielding zone is configured as a sealing area for preventing an external object from entering, which can effectively reduce a pollution situation possibly generated due to not sterilizing the total-shadow shielding zone, to ensure the safety and accuracy of detection.

The above summary is merely for the purpose of specification, and not intended to make a limitation in any way. In addition to the schematic aspects, implementation modes and features described above, the further aspects, implementation modes and features of the present disclosure will be easily understood with reference to drawings and the detailed description below.

### BRIEF DESCRIPTION OF THE DRAWINGS

In the drawings, unless otherwise specified, the same drawing signs penetrating through a plurality of drawings indicate the same or similar parts or elements. These drawings are not necessarily drawn to scale. It should be understood that these drawings only describe some implementation modes of the present disclosure, and should not be regarded as limiting the scope of the present disclosure.

FIG. 1 is an exploded view of a detector according to a first embodiment of the present disclosure.

FIG. 2 is a top view of a detector according to embodiments of the present disclosure.

FIG. 3 is a profile diagram along A-A in FIG. 2.

FIG. 4 is a profile diagram along B-B in FIG. 2.

FIG. 5 is a structural schematic diagram of a first barrel-shaped bracket of a detector according to embodiments of the present disclosure.

FIG. 6 is a structural schematic diagram of a second barrel-shaped bracket of a detector according to embodiments of the present disclosure.

FIG. 7 is a structural schematic diagram of a first bracket of a detector according to embodiments of the present disclosure.

FIG. 8 is a schematic diagram of a total-shadow shielding zone of a detector according to embodiments of the present disclosure.

FIG. 9 is a profile diagram of a detector according to a second embodiment of the present disclosure.

FIG. 10 is a structural schematic diagram of a detector according to a third embodiment of the present disclosure.

FIG. 11 is a structural schematic diagram of a detector according to a fourth embodiment of the present disclosure.

FIG. 12 is a structural schematic diagram of a detector according to a fifth embodiment of the present disclosure.

FIG. 13 is a structural schematic diagram of a detection circuit board of a detector according to a sixth embodiment of the present disclosure.

FIG. 14 is a structural schematic diagram of a total-shadow shielding zone formed by a shielding frame in FIG. 13.

FIG. 15 is a structural schematic diagram of a total-shadow shielding zone formed by a shielding frame of a detector according to a seventh embodiment of the present disclosure.

FIG. 16 is a structural schematic diagram of a detection circuit board of a detector according to an eighth embodiment of the present disclosure.

FIG. 17 is a structural schematic diagram of a total-shadow shielding zone formed by a shielding frame in FIG. 16.

FIG. 18 is a structural schematic diagram of a shielding frame of a detector according to a ninth embodiment of the present disclosure.

FIG. 19 is a structural schematic diagram of a shielding frame of a detector according to a tenth embodiment of the present disclosure.

FIG. 20 is a solid diagram of a detector according to an eleventh embodiment of the present disclosure.

FIG. 21 is an exploded view of a detector according to an eleventh embodiment of the present disclosure.

FIG. 22 is an exploded view in another angle of a detector according to an eleventh embodiment of the present disclosure.

FIG. 23 is a partial structural schematic diagram of a detector according to an eleventh embodiment of the present disclosure.

FIG. 24 is a profile diagram along a line C-C in FIG. 20.

FIG. 25 is a profile diagram along a line D-D in FIG. 20.

FIG. 26 is a profile diagram along a line E-E in FIG. 20.

FIG. 27 is a partial structural schematic diagram of a detector according to an eleventh embodiment of the present disclosure.

FIG. 28 is a schematic diagram of an outer package of a detector according to embodiments of the present disclosure.

FIG. 29 is an exploded view of an outer package shown in FIG. 28.

FIG. 30 is an exploded view in another angle of an outer package shown in FIG. 28.

FIG. 31 is a schematic diagram of an outer package of a detector according to another embodiment of the present disclosure.

FIG. 32 is a schematic diagram of a detector according to another embodiment of the present disclosure.

FIG. 33 is a schematic diagram of a partial structure of a detector according to a twelfth embodiment of the present disclosure.

FIG. 34 is a three-dimensional exploded view of the detector shown in FIG. 33.

FIG. 35 is a schematic diagram of a partial structure of a detector according to a twelfth embodiment of the present disclosure.

FIG. 36 is a three-dimensional diagram of a control module of a detector according to a twelfth embodiment of the present disclosure.

FIG. 37 is a three-dimensional exploded view of the control module the detector shown in FIG. 36.

FIG. 38 is a schematic structural diagram of the control module the detector shown in FIG. 36 in another view.

FIG. 39 is a schematic structural diagram of a shielding assembly of the detector shown in FIG. 36.

FIG. 40 is a schematic structural diagram of a second antenna of the detector shown in FIG. 36.

FIG. 41 is a schematic structural diagram of a battery holder of the detector shown in FIG. 36.

FIG. 42 is a schematic structural diagram of a first antenna of the detector shown in FIG. 36.

FIG. 43 is a schematic structural diagram of the first antenna shown in FIG. 42 in other embodiments.

FIG. 44 is a schematic structural diagram of the first antenna shown in FIG. 42 in another embodiment.

FIG. 45 is a circuit diagram of a matching network of the detector shown in FIG. 36.

FIG. 46 is a circuit diagram of the control module the detector shown in FIG. 36.

FIG. 47 is a schematic diagram of a partial structure of a detector according to a thirteenth embodiment of the present disclosure.

FIG. 48 is a three-dimensional exploded view of the detector shown in FIG. 47.

FIG. 49 is a schematic diagram of a partial structure of a detector according to a fourteenth embodiment of the present disclosure.

FIG. 50 is a three-dimensional diagram of an electric control module of a detector according to a fourteenth embodiment of the present disclosure.

FIG. 51 is a three-dimensional exploded view of an electric control module of the detector shown in FIG. 50.

FIG. 52 is a circuit diagram of the electric control module the detector shown in FIG. 50.

FIG. 53 is a circuit diagram of a matching network unit in the electric control module of the detector shown in FIG. 50.

FIG. 54 is a schematic structural diagram of a sterilization system according to an embodiment of the present disclosure.

FIG. 55 is a schematic structural diagram of a bearing frame of a sterilization system according to an embodiment of the present disclosure.

FIG. 56 is a cross-sectional view of a bearing frame of a sterilization system according to an embodiment of the present disclosure.

FIG. 57 is another schematic structural diagram of a sterilization system according to an embodiment of the present disclosure.

FIG. 58 is another schematic structural diagram of a bearing frame of a sterilization system according to an embodiment of the present disclosure.

FIG. 59 is still another schematic structural diagram of a bearing frame of a sterilization system according to an embodiment of the present disclosure.

FIG. 60 is another partially structural exploded view of another structure of a sterilization system according to an embodiment of the present disclosure.

FIG. 61 is an exploded view of a partial structure of the sterilization system shown in FIG. 60 in another angle.

FIG. 62 is a schematic diagram of the bearing frame of the sterilization system shown in FIG. 60.

FIG. 63 is a schematic diagram of a cover plate of the sterilization system shown in FIG. 60.

FIG. 64 is a schematic structural diagram of the sterilization system shown in FIG. 60.

FIG. 65 is a schematic diagram of combination of the bearing frame and the cover plate of the sterilization system shown in FIG. 60.

### DETAILED DESCRIPTION OF THE EMBODIMENTS

Only some exemplary embodiments are simply described below. Just as those skilled in the art may understand, the described embodiments may be modified by various different modes without departing from the spirit or scope of the present disclosure. Therefore, drawings and description are regarded as being essentially exemplary, instead of being restrictive.

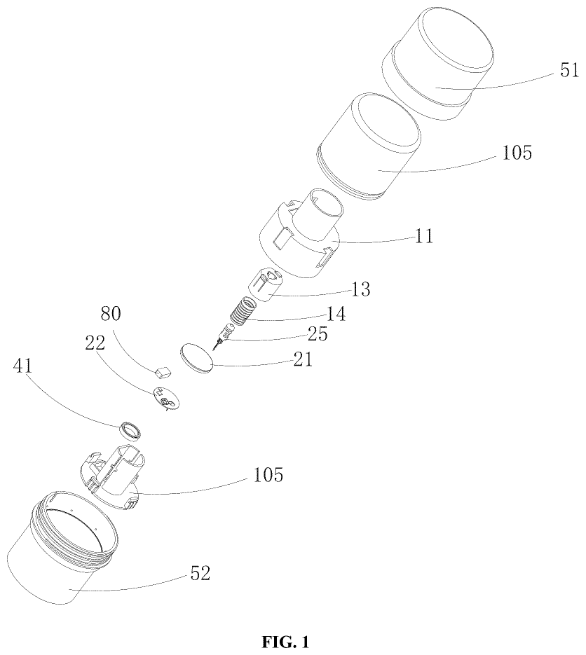

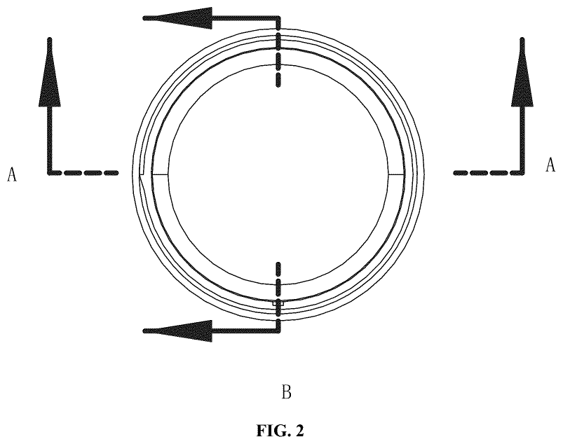

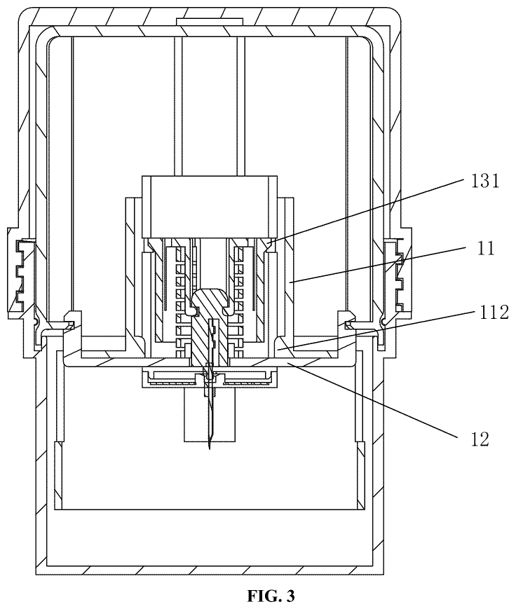

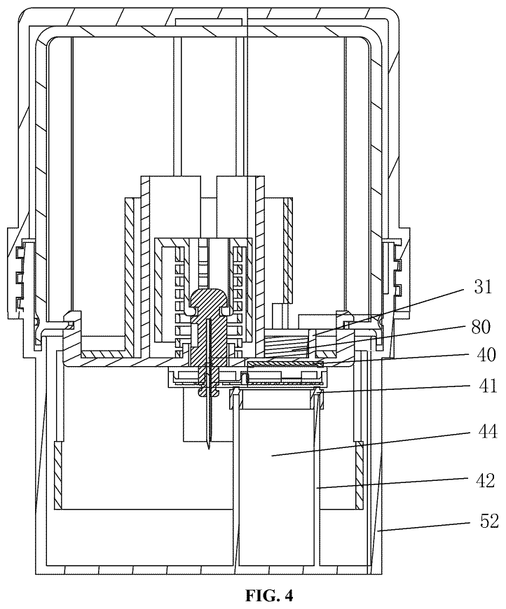

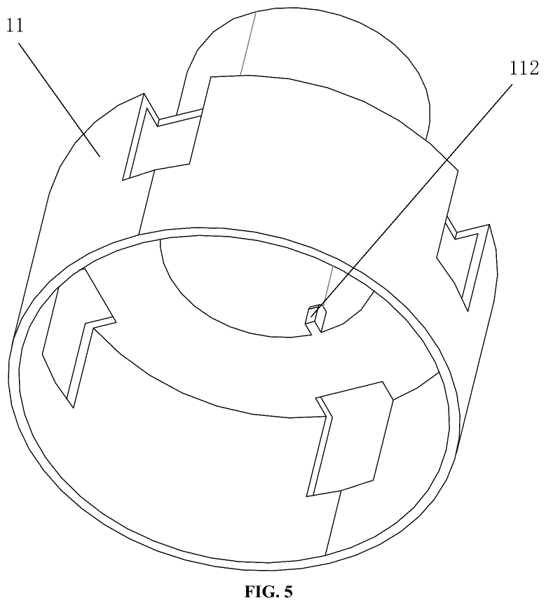

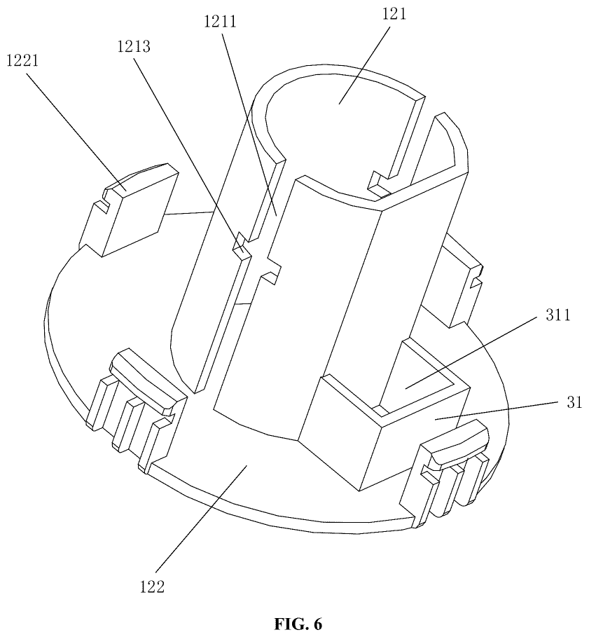

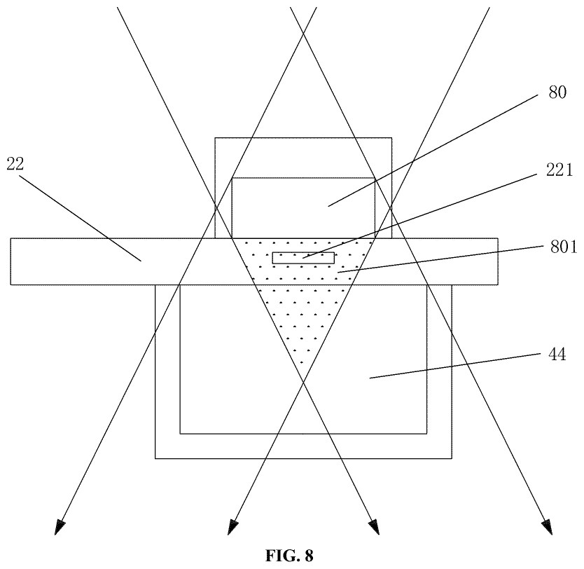

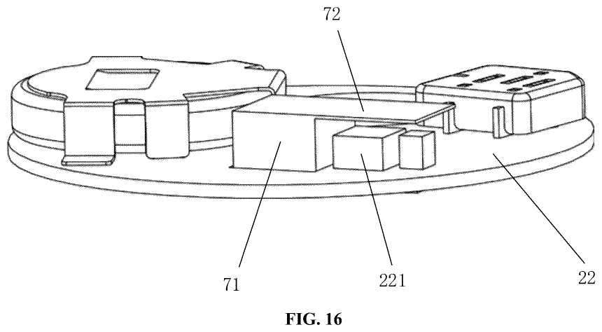

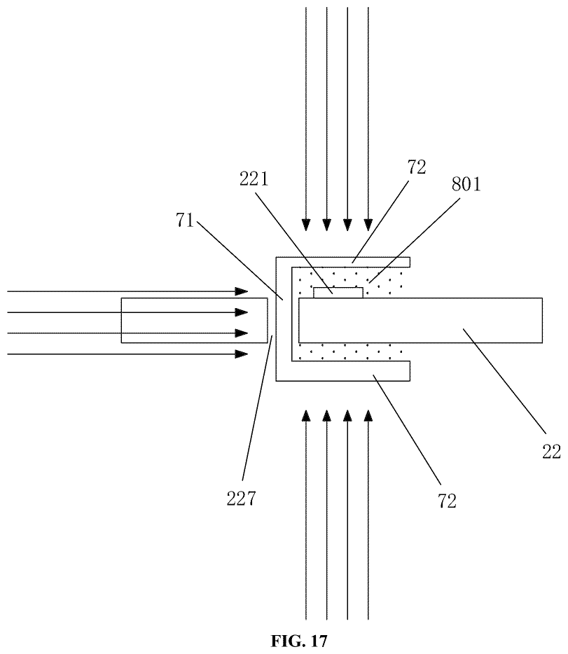

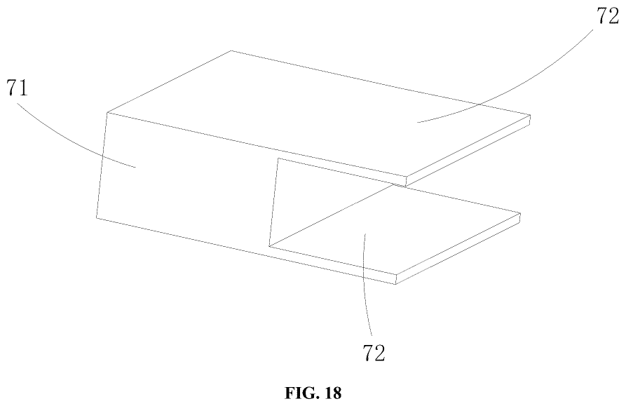

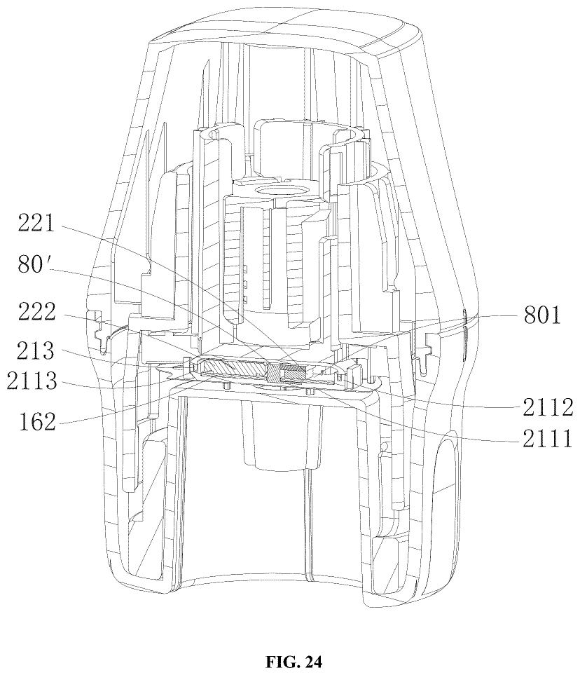

Please refer to FIGS. 1-24, embodiments of the present disclosure provide a detector, and the detector includes a housing assembly, a detection assembly and a shielding assembly, and detailed descriptions are made below.

The detection assembly includes a first housing 21, a detection circuit board 22 and a probe (not shown in the drawings), the detection circuit board 22 is disposed in the first housing 21 and electrically connected with a first end of the probe, the detection circuit board 22 includes a sensitive element 221, and the first end of the probe is fixed in the first housing 21 while a second end stretches out of the first housing 21;
the housing assembly includes a collision housing and a pressing portion that can slide in relative to each other, the detection assembly is located below a bottom of the pressing portion, the collision housing is used for abutting against a sampling part, the pressing portion is used for driving the detection assembly to move towards the sampling part, so as to pierce the probe into the sampling part, and a detection signal of the probe is transmitted to the detection circuit board 22; and
the shielding assembly is disposed on one side of the first housing 21, when the detector is sterilized through an irradiation ray, the shielding assembly is used for blocking part of the irradiation ray, to form a total-shadow shielding zone 801 for protecting the sensitive element 221. The at least part of the total-shadow shielding zone 801 may be configured as a sealing area for preventing an external object from entering, thus effectively reducing a pollution situation possibly generated due to not sterilizing the total-shadow shielding zone 801, to ensure the safety and accuracy of detection. The external object may be external gas, dust, moisture and the like that may carry bacteria.

The sensitive element 221 is a high-precision electronic component that is easily affected by the irradiation ray and breaks down.

The irradiation ray includes but is not limited to an X-ray, an electronic beam and a gamma ray.

When the irradiation ray is emitted to the sensitive element 221 on the detection circuit board 22 from one side of the first housing 21, and the shielding assembly can block the irradiation ray emitted to the sensitive element 221 and form the total-shadow shielding zone 801 not affected by the irradiation ray; and adjusting the position and size of the shielding assembly enables the total-shadow shielding zone 801 formed by the shielding assembly to cover the sensitive element 221, and weakens or eliminates the irradiation influence on the sensitive element 221 by the irradiation ray, thus relieving a situation that the detection circuit board 22 cannot work normally due to a failure of the sensitive element 221 affected by the irradiation. That is, the present disclosure can also protect the detection circuit board 22 from being damaged in a case of guaranteeing the irradiation ray to sterilize the detector.

In this embodiment, the detector may be disposed below the irradiation source when being sterilized, such that the total-shadow shielding zone 801 formed by the shielding assembly wraps the sensitive element 221.

Specifically, a section of the total-shadow shielding zone 801 formed by the shielding assembly is a triangle or a polygon.

Please refer to FIG. 1 to FIG. 6, in one alternative embodiment, the probe is brought into the sampling part through the guide needle 25;
the collision housing includes a first barrel-shaped bracket 11;
the pressing portion includes a pressing shell 105, a second barrel-shaped bracket 12, a support bracket 13 and an elastic element 14;
the first barrel-shaped bracket 11 is slidingly and partially located in the pressing shell 105, and an inner wall of the first barrel-shaped bracket 11 is provided with a raised portion 112;
the second barrel-shaped bracket 12 is slidingly sleeved in the first barrel-shaped bracket 11, the second barrel-shaped bracket 12 includes a barrel-shaped structure 121 and a base plate 122 at a bottom thereof, the barrel-shaped structure 121 is provided with a guide hole 1211 along an axial direction, a positioning hole 1213 is provided on a path of the guide hole 1211, a plurality of connecting portions 1221 extend on an outer edge of the base plate 122, and the plurality of connecting portions 1221 are fixedly connected with the pressing shell 105;
the support bracket 13 is located in the barrel-shaped structure 121, and an elastic resisting portion 131 extends outside the support bracket 13;
the elastic element 14 is compressed between the base plate 122 and a top of the support bracket 13;
in an initial state, the elastic element 14 is located in a compressed state, and the elastic resisting portion 131 is located in the positioning hole 1213; and
under the action of an external force, the pressing shell 105 drives the second barrel-shaped bracket 12 to move in relative to the first barrel-shaped bracket 11, such that the raised portion 112 moves along the guide hole 1211; when the second barrel-shaped bracket 12 moves to a first preset position in relative to the first barrel-shaped bracket 11, the guide needle 25 guides the probe to be pierced into the sampling part; and when the second barrel-shaped bracket 12 moves to a second preset position in relative to the first barrel-shaped bracket 11, the raised portion 112 extrudes the elastic resisting portion 131 out of the positioning hole 1213, the elastic element 14 is released, and the support bracket 13 drives the guide needle 25 to leave the sampling part.

In this embodiment, the elastic element 14 may be a spring. Since the elastic element 14 is compressed between the base plate 122 and the top of the support bracket 13, that is, the elastic element 14 will apply an elastic force to the second barrel-shaped bracket 12 and the support bracket 13 to enable the barrel-shaped bracket 12 to be away from the support bracket 13, but the elastic resisting portion 131 is located in the positioning hole 1213, the elastic force generated by the elastic element 14 will push the elastic resisting portion 131 to abut against the inner wall of the positioning hole 1213, such that the second barrel-shaped bracket 12 and the support bracket 13 are stationary relatively.

Specifically, the detector may be used for detecting a plurality of body index data, and the following embodiments take the detection for blood glucose as an example.

When a user adopts the detector for detecting the blood glucose, the user holds the pressing shell 105 to align with the sampling part, such that one end of the collision shell abuts against a vicinity of the sampling portion, at this time the guide needle 25 is not in contact with the sampling part, and then the pressing shell 105 is pushed to move towards one side closing to the sampling part; since the pressing shell 105 is connected with the second barrel-shaped bracket 12, the support bracket 13 also abuts against the second barrel-shaped bracket 12 through the elastic resisting portion 131, the pressing shell 105 can drive the support bracket 13 and the detection assembly to move towards one side closing to the sampling part when driving the second barrel-shaped bracket 12 to move. In a process that the second barrel-shaped bracket 12 drives the detection assembly to move, the guide needle 25 in the detection assembly approaches to the sampling part gradually until the second barrel-shaped bracket 12 moves to the first preset position, and the guide needle 25 is pierced into the sampling part. The probe is pierced along the guide needle 25, thus sampling through the probe, and the probe transmits the data to the detection circuit board 22 to detect blood glucose indexes.

The user continues to push the pressing shell 105 to move towards one side closing to the sampling part until the second barrel-shaped bracket 12 reaches the second preset position. At this time, the raised portion 112 extrudes the elastic resisting portion 131 out of the positioning hole 1213, the elastic resisting portion 131 does not abut against the hole wall of the positioning hole 1213 any more, the support bracket 13 is not limited by the second barrel-shaped bracket 12 any more, the elastic force generated by the compressed elastic element 14 will push the support bracket 13 towards one side away from the sampling part, thus driving the guide needle 25 leave the sampling part, and quickly pulling out the guide needle 25 from the sampling part. Since the detection assembly is not connected with the support bracket 13, the probe is not pulled out of the sampling part together with the guide needle 25, instead of remaining in the sampling part for continuous sampling detection, thus dynamically feeding back a detection result.

The user may push the pressing shell 105 through the structure of the above-mentioned detector, the probe is pierced into the sampling part through the guide needle 25, the support bracket 13 is not limited by the second barrel-shaped bracket 12 any more after the guide needle 25 brings the probe into the sampling part for a certain depth, the spring is released such that the guide needle 25 is quickly pulled out of the sampling part along the piercing path, so situations of strong pain, skin injury and the like of to-be-detected personnel, caused by deviating from the trajectory, relatively slow needle withdrawal and the like, do not appear easily, thus improving the experience of the to-be-detected personnel.

Further, in this embodiment, the shielding assembly includes a shielding block fixed structure 31 and a shielding block 80;
the shielding block fixed structure 31 is fixed above the base plate 122, and a shielding block accommodating slot 311 is provided on the shielding block fixed structure; 31 and
the shielding block 80 is removably accommodated in the shielding block accommodating slot 311, to block the irradiation ray and form the total-shadow shielding zone 801 in a designated area.

It can be understood that after irradiation sterilization for the detector is completed, the shielding block 80 may be taken out of the shielding block accommodating slot 311, and may also be kept in the detector.

Specifically, the shielding block 80 is a cuboid structure. The shielding block 80 has a density greater than 1,000 kg/m³. It can be understood that the greater the density of the shielding block 80, the better the effect that the shielding block 80 blocks the irradiation ray.

It is to be noted that although the shielding block 80 can block the irradiation ray, the sterilization strength to the total-shadow shielding zone 801 by the irradiation ray is also weakened, the insufficient sterilization strength may appear, thus not sterilizing the total-shadow shielding zone 801 completely, leading to bacteria still existing in the total-shadow shielding zone 801 in the detector after the irradiation sterilization, thereby polluting other areas of the detector.

Based on the above-mentioned issues, in this embodiment, the detector further includes a sealing shell 40, the sealing shell 40 is located between the base plate 122 and the first housing 21, the sealing shell 40 and the outer wall of the first housing 21 form a first sealing cavity (not shown in the drawings), and the first sealing cavity is disposed correspondingly to the shielding assembly. It can be understood that the total-shadow shielding zone 801 includes an area where part of the first sealing cavity is located. The sensitive element can be disposed in the first sealing cavity.

Preferably, a cross section of the first sealing cavity is greater than or equal to that of the total-shadow shielding zone formed by the shielding assembly.

A gap exists between the base plate 122 and the first housing 21, while the gap is also located in the total-shadow shielding zone 801, therefore setting the sealing shell 40 between the base plate 122 of the second barrel-shaped bracket 12 and the first housing 21 may wrap a gap therebetween into the first sealing cavity, thus avoiding a situation that residual bacteria possibly exists in the gap therebetween due to the irradiation ray weakened by the shielding block 80, then other areas of the detector are polluted.

Further, in this embodiment, the detector further includes a sealing structure, the sealing structure is located on one side of the shielding assembly that is away from the first sealing cavity, and the sealing structure has a second sealing cavity 44. It can be understood that the total-shadow shielding zone 801 includes the area in which at least part of the second sealing cavity 44 is located, and specifically the second sealing cavity 44 is used for wrapping the at least part of the total-shadow shielding zone 801, that is, the area where the second sealing cavity 44 is located may be equal or or exceed the total-shadow shielding zone 801.

The at least part of the total-shadow shielding zone 801 is wrapped through the second sealing cavity 44, such that the bacteria possibly remaining in the total-shadow shielding zone 801 is isolated from other areas of the detector, thus avoiding the situations that the sterilization strength to the total-shadow shielding zone 801 is insufficient due to the irradiation ray weakened by the shielding block 80, then other areas of the detector are polluted.

Further, the detector further includes a packaging assembly, the packaging assembly includes a first outer shell 51 and a second outer shell 52, and the first outer shell 51 is mutually coupled with the second outer shell 52 to wrap the housing assembly;
the sealing structure includes a first bracket 41 and a second bracket 42, the first bracket 41 is fixed to the first housing 21, and the second bracket 42 is fixed to the second outer shell 52; and
in a case that the first outer shell 51 is coupled with the second outer shell 52, the first bracket 41 cooperates with the second bracket 42 to form the second sealing cavity 44.

When the user unpacks the detector for use, the second bracket 42 is removed together with the second outer shell 52 since the second bracket 42 is fixedly connected with the second outer shell 52, thus avoiding the second bracket 42 interfering the use of the user. However, at this time the user has unpacked the detector, the second sealing cavity 44 is opened, but the detector has been in contact with the outside world and used, so there is no problem that the bacteria in the second sealing cavity 44 pollute other areas of the detector.

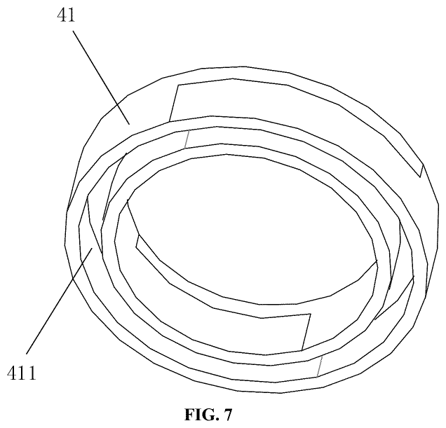

Further, please combine with FIGS. 1-6 and refer to FIG. 7, the first bracket 41 is a hollow cylinder, and one end of the first bracket 41 is hermetically connected with the first housing 21 while the other end is provided with a fitting groove 411;
the second bracket 42 is a hollow cylinder, and one end of the second bracket 42 that is away from the first bracket 41 is fixedly connected with the second outer shell 52; and
in a case that the first outer shell 51 is coupled with the second outer shell 52, one end of the second bracket 42 that is away from the second outer shell 52 is embedded into the fitting groove 411, to form the second sealing cavity 44 together with the first bracket 41.

Specifically, the first bracket 41 may be made of a flexible material, including but not being limited to silica gel, thermoplastic polyurethane (TPU), rubber, plastics, etc. The first bracket 41 made of the flexible material can be hermetically connected with the second bracket 42 more tightly, to ensure the connecting tightness therebetween.

Alternatively, in this embodiment, the first outer shell 51 is in threaded connection with the second outer shell 52.

On the one hand, the threaded connection between the first outer shell 51 and the second outer shell 52 may enable the detector to fit with the packaging assembly more tightly, thus avoiding accidental unpacking caused by shaking during transportation; and on the other hand, in a process that the detector is packed into the packaging assembly, since the second bracket 42 is fixed to the second outer shell 52, a pretightening force can be provided to the bracket when tightening the first outer shell 51 and the second outer shell 52, such that the second bracket 42 is closer to the first bracket 41 and the second bracket 42 fits with the first bracket 41 more tightly, thus ensuring the tightness of the bracket.

Further, the detector further includes a shielding block placement structure, and the shielding block placement structure is used for placing the shielding block 80 removably; and
in a case that the shielding block 80 is placed on the shielding block placement structure, the shielding block 80 is used for preventing the irradiation ray from being emitted to the sensitive element 221.

When the detector is sterilized through the irradiation, the shielding block 80 is placed on the shielding block placement structure of the detector, to further protect the sensitive element 221 from being affected by the irradiation.

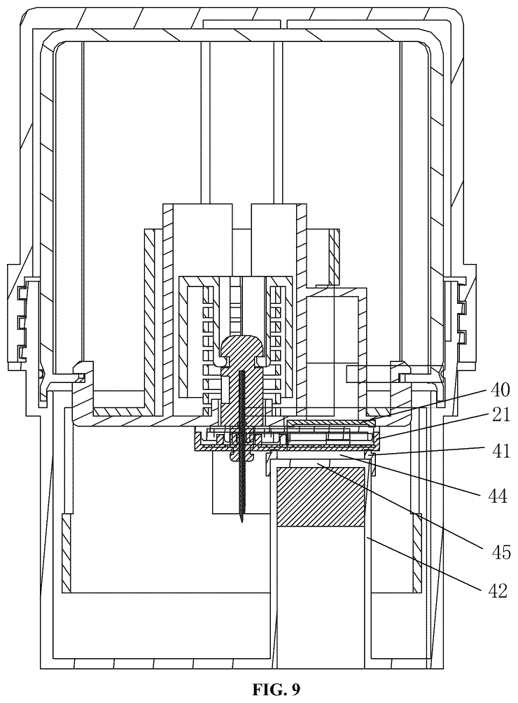

In other alternative embodiments, as shown in FIG. 9, the detector further includes a packaging assembly, the packaging assembly includes a first outer shell 51 and a second outer shell 52, and the first outer shell 51 is mutually coupled with the second outer shell 52 to wrap the housing assembly;
the detector further includes a sealing structure, and the sealing structure is located on one side of the first housing 21 that is away from the shielding assembly; and the sealing structure has a second sealing cavity, the second sealing cavity is used for wrapping at least part of the total-shadow shielding zone, the sealing structure includes a first bracket 41 and a second bracket 42, the first bracket 41 is fixed to the first housing 21, and the second bracket 42 is fixed to the second outer shell 52;
the sealing structure further includes an isolating plate 45, the isolating plate 45 is disposed in the second bracket 42, and the isolating plate 45 is hermetically connected with the inner wall of the second bracket 42;
a via hole is provided on the second outer shell 52, and the via hole is disposed correspondingly to the second bracket 42; and
the second bracket 42 and the isolating plate 45 form the shielding block placement structure, and in a case that the shielding block 80 is placed in the shielding block placement structure, the shielding block 80 forms the total-shadow shielding zone for wrapping the sensitive element.

The shielding block 80 is placed in the second bracket 42 through the via hole, and the shielding block 80 is enabled to abut against the isolating plate 45 to shield the irradiation ray. The shielding block 80 in the second bracket 42 is disposed in relative to the shielding block 80 in the shielding assembly on the other side of the first housing 21, to block the irradiation rays on both sides of the sensitive element 221, respectively.

After irradiation sterilization, the shielding block 80 can be directly taken out of the second bracket 42 through the via hole, such that the shielding block 80 is mounted in another detector when the another detector is sterilized, to achieve the reuse of the shielding block 80.

In other alternative embodiments, a penetrating hole is provided on an end face of the first outer shell;
an end face of the second outer shell depresses inward to form a first depressed portion;
correspondingly, an end face of the housing assembly that is close to the first outer shell depresses inward to form a second depressed portion; and
the penetrating hole, first depressed portion and second depressed portion are disposed correspondingly and combined to form the shielding block placement structure.

When the shielding block is placed on the shielding block placement structure, the shielding block is located on two opposite sides of the sensitive element to form the total-shadow shielding zone for wrapping the sensitive element.

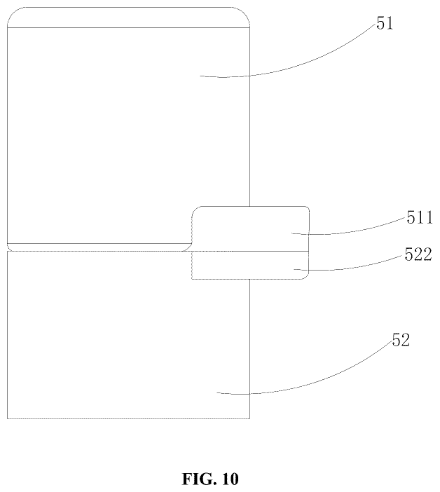

In another alternative embodiment, as shown in FIG. 10, a first raised shell 511 is provided on the first outer shell 51, a second raised shell 522 is provided on the second outer shell 52, the first raised shell 511 cooperates with the second raised shell 522 to form a raised cavity (not shown in the drawings), and the raised cavity is used for accommodating the sensitive element 221 of the detection circuit board 22 in the first housing 21;
an outer wall of the first raised shell 511 and an outer wall of the second raised shell 522 are used for forming the shielding block placement structure.

In this embodiment, the corresponding part of the high-precision electronic component in the detection circuit board 22 protrudes from the housing assembly, and the first raised shell 511 and the second raised shell 522 accommodate part of the detection circuit board 22. In this embodiment, the shielding block 80 can be disposed at an upper end of the first raised shell 511 and a lower end of the second raised shell 522, and sides of the first raised shell 511 and the second raised shell 522, such that the shielding block 80 blocks the irradiation ray generated during irradiation sterilization.

In another alternative embodiment, as shown in FIG. 11, a waist portion of the packaging assembly depresses inward to form a third depressed portion 54, and the third depressed portion 54 is disposed correspondingly to the first housing 21; and
the third depressed portion 54 is used for forming the shielding block placement structure.

The shielding block 80 is disposed on the third depressed portion 54 at the waist portion of the first housing 21, such that the shielding block 80 is located on the path that the irradiation ray is emitted to the sensitive element 221, thus reducing the situation that the sensitive element 221 breaks down caused when the irradiation ray is emitted to the side of the sensitive element 221.

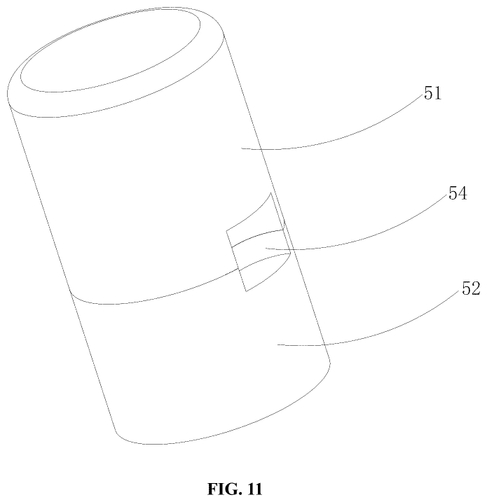

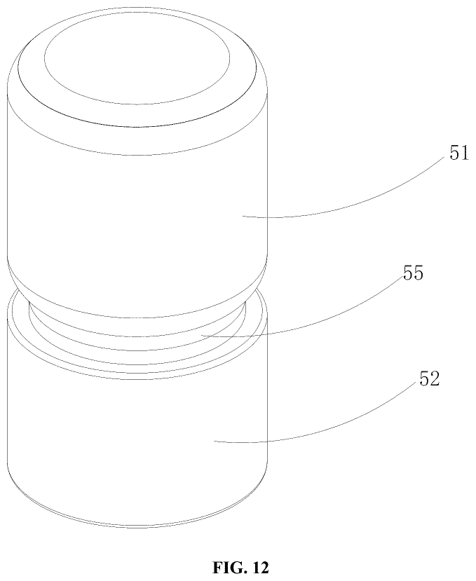

In another alternative embodiment, as shown in FIG. 12, a waist portion of the packaging assembly depresses inward to form an annular groove 55, the annular groove 55 is disposed correspondingly to the first housing 21; and
the annular groove 55 is used for forming the shielding block placement structure.

The shielding block 80 is disposed on the annular groove 55 at the waist portion of the first housing 21, such that the irradiation ray from any side end of the sensitive element 221 can be blocked, the shielding block 80 is located on the path that the irradiation ray is emitted to the sensitive element 221, thus reducing the influence on the sensitive element 221 by the irradiation ray.

In one alternative embodiment, the guide needle 25 is fixedly connected with the support bracket 13, and one end of the guide needle 25 passes through the detection assembly; and
the detector further includes a needle sleeve, the needle sleeve is used for accommodating the guide needle 25, and one end of the needle sleeve is hermetically connected with the first housing 21.

Sterilization from the irradiation ray is received throughout the needle sleeve, thus not existing bacteria. The guide needle 25 is accommodated in the needle sleeve, such that the guide needle 25 is isolated from other spaces of the detector, to avoid a situation that the shielding block 80 blocks the irradiation ray, leading to the insufficient sterilization strength and then causing the guide needle 25 to be polluted by the residual bacteria.

In other alternative embodiments, the other end of the needle sleeve is fixedly connected with the second outer shell 52.

In other alternative embodiments, the detector further includes a shielding sheet, the shielding sheet is disposed in the first housing, the shielding sheet is located on a path that the irradiation ray is emitted to the sensitive element, and forms the total-shadow shielding zone for wrapping the sensitive element.

Specifically, the shielding sheet may be located above the sensitive element 221 to form the total-shadow shielding zone to cover the sensitive element 221.

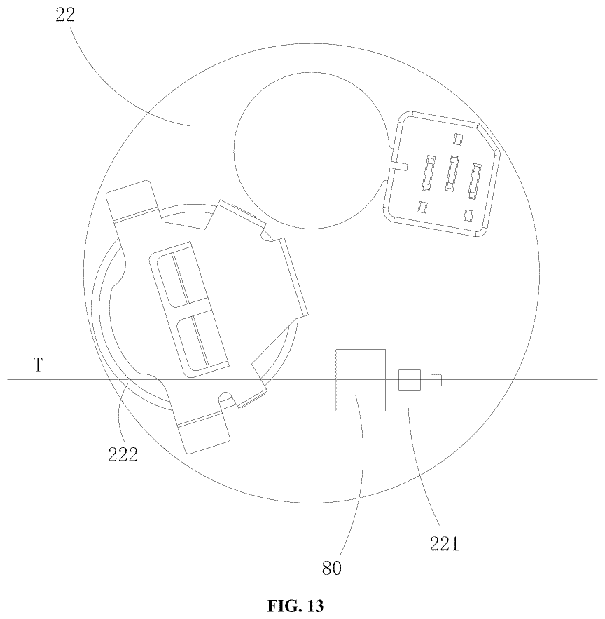

Please combine with FIGS. 1-12 and refer to FIGS. 13-19, embodiments of the present disclosure provide a detector, and the detector includes a housing assembly, a detection assembly and a shielding assembly, where:
the detection assembly includes a first housing 21, a detection circuit board 22 and a probe, the detection circuit board 22 is disposed in the first housing 21 and electrically connected with a first end of the probe, the detection circuit board 22 includes a sensitive element 221, and the first end of the probe is fixed in the first housing 21 while a second end stretches out of the first housing 21;
the housing assembly includes a collision housing and a pressing portion that can slide in relative to each other, the detection assembly is located below a bottom of the pressing portion, the collision housing is used for abutting against a sampling part, the pressing portion is used for driving the detection assembly to move towards the sampling part, so as to pierce the probe into the sampling part, and a detection signal of the probe is transmitted to the detection circuit board 22; and
the shielding assembly is disposed in the first housing 21, when the detector is sterilized through an irradiation ray, the shielding assembly is used for blocking part of the irradiation ray, to form a total-shadow shielding zone 801 for protecting the sensitive element 221. Further, as shown in FIG. 13, the detection circuit board 22 is also provided with a battery module 222, the battery module 222, the shielding assembly and the sensitive element 221 are all disposed on the path that the irradiation source for sterilization is emitted to the detector, such that the battery module 222 and shielding assembly block the radiation of the irradiation source to be emitted to the sensitive element 221 together.

The difference from the above-mentioned embodiment is that the shielding assembly in the detector in this embodiment is disposed in the first housing 21.

When the irradiation ray is emitted to the sensitive element 221 on the detection circuit board 22 from one side of the first housing 21, and the shielding assembly can block the irradiation ray emitted to the sensitive element 221 and form the total-shadow shielding zone 801 not affected by the irradiation ray; and adjusting the position and size of the shielding assembly enables the total-shadow shielding zone 801 formed by the shielding assembly to cover the sensitive element 221, and weakens or eliminates the irradiation influence on the sensitive element 221 by the irradiation ray, thus relieving a situation that the detection circuit board 22 cannot work normally due to a failure of the sensitive element 221 affected by the irradiation. That is, the present disclosure can also protect the detection circuit board 22 from being damaged in a case of guaranteeing the irradiation ray to sterilize the detector.

After being emitted from an accelerator, the electronic beam passes through non-vacuum substances in various parts such as air, sensor outer surface and outer shell; and these substances form a strong scattering action on electron, but the electron cannot penetrate through the shielding assembly, and will form a low-radiation area below the shielding assembly, that is, the total-shadow shielding zone. Due to different electronic scattering angles, an edge of the total-shadow shielding zone also has a radiation dosage, and the radiation dosage in the area from the edge of the total-shadow shielding zone to the center and the upper part closing to the shielding assembly is relatively small, and the dosage further away from the shielding assembly is relatively great. By adjusting the structure and position of the shielding assembly, the area with the relatively small radiation dosage in the total-shadow shielding zone formed by the shielding assembly wraps the sensitive element.

Further, the probe is brought into the sampling part through the guide needle 25;
the collision housing includes a first barrel-shaped bracket 11;
the pressing portion includes a pressing shell 105, a second barrel-shaped bracket 12, a support bracket 13 and an elastic element 14;
the first barrel-shaped bracket 11 is slidingly and partially located in the pressing shell 105, and an inner wall of the first barrel-shaped bracket 11 is provided with a raised portion 112;
the second barrel-shaped bracket 12 is slidingly sleeved in the first barrel-shaped bracket 11, the second barrel-shaped bracket 12 includes a barrel-shaped structure 121 and a base plate 122 at a bottom thereof, the barrel-shaped structure 121 is provided with a guide hole 1211 along an axial direction, a positioning hole 1213 is provided on a path of the guide hole 1211, a plurality of connecting portions 1221 extend on an outer edge of the base plate 122, and the plurality of connecting portions 1221 are fixedly connected with the pressing shell 105;
the support bracket 13 is located in the barrel-shaped structure 121, and an elastic resisting portion 131 extends outside the support bracket 13;
the elastic element 14 is compressed between the base plate 122 and a top of the support bracket 13;
in an initial state, the elastic element 14 is located in a compressed state, and the elastic resisting portion 131 is located in the positioning hole 1213; and

under the action of an external force, the pressing shell 105 drives the second barrel-shaped bracket 12 to move in relative to the first barrel-shaped bracket 11, such that the raised portion 112 moves along the guide hole 1211; when the second barrel-shaped bracket 12 moves to a first preset position in relative to the first barrel-shaped bracket 11, the guide needle 25 guides the probe to be pierced into the sampling part; and when the second barrel-shaped bracket 12 moves to a second preset position in relative to the first barrel-shaped bracket 11, the raised portion 112 extrudes the elastic resisting portion 131 out of the positioning hole 1213, the elastic element 14 is released, and the support bracket 13 drives the guide needle 25 to leave the sampling part.

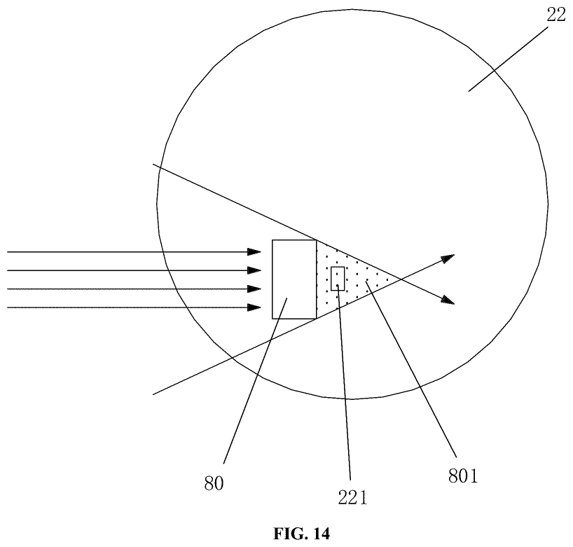

In one alternative embodiment, as shown in FIG. 13 and FIG. 14, the shielding assembly includes a shielding frame, the shielding frame is fixed to one side of the detection circuit board 22 that is provided with the sensitive element 221, and the shielding frame is used for blocking the irradiation ray to form the total-shadow shielding zone 801 in the designated area.

When being sterilized, the detector can be transversely placed, the position of the detector is adjusted such that the shielding frame is located on the path that the irradiation ray is emitted to the sensitive element 221, and the total-shadow shielding zone 801 formed by the shielding frame protects the sensitive element 221 from being affected by the irradiation ray.

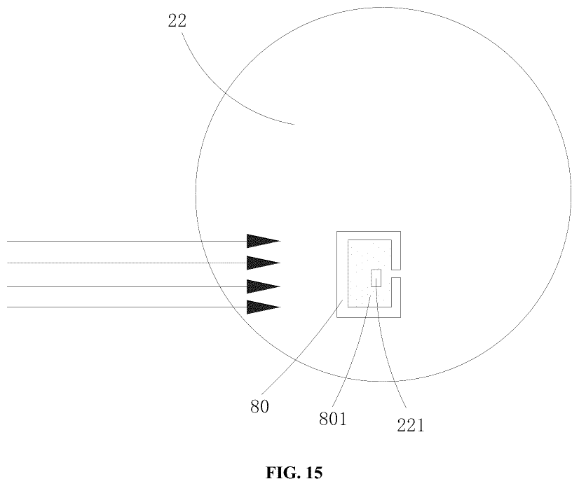

In one alternative embodiment, as shown in FIG. 15, the shielding assembly includes a shielding frame, the shielding frame is fixed to the detection circuit board 22 and disposed around a periphery of the sensitive element 221, and the shielding frame is used for blocking the irradiation ray to form the total-shadow shielding zone 801 in the designed area.

When being sterilized, the detector can be transversely placed, since the shielding frame is disposed around the periphery of the sensitive element 221, the space in the shielding frame is all the total-shadow shielding zone 801, which can block the irradiation ray emitted from any direction of the side end of the sensitive element 221, to protect the sensitive element 221 from being affected by the irradiation ray.

In one alternative embodiment, please refer to FIGS. 16-18, a fixed hole 227 is provided on the detection circuit board 22; and
the shielding assembly includes a shielding frame, the shielding frame includes a connecting plate 71a and two side baffles 72, the connecting plate 71 is worn in the fixed hole 227, and the two side baffles 72 are separately fixed to the two opposite ends of the connecting plate 71 to clamp the sensitive element 221.

When being sterilized, the detector can be placed below the irradiation source, the sensitive element 221 is located between the two side baffles 72, the connecting plate 71 is located at one end of the sensitive element 221, such structure can at least block the irradiation rays from three directions of the sensitive element 221, and the total-shadow shielding zone 801 formed by the two side baffles 72 and the connecting plate 71 covers the sensitive element 221, to protect the sensitive element 221 from being affected by the irradiation ray.

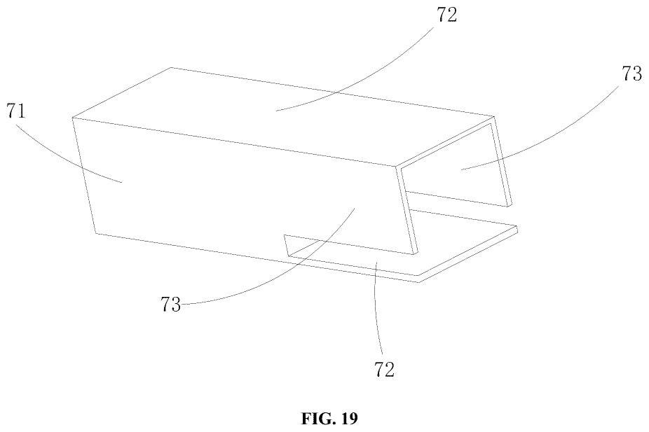

Further, as shown in FIG. 19, the shielding frame further includes vertical plates 73, the vertical plates 73 are located on one side of the detection circuit board 22 that is provided with the sensitive element 221, and one end of each of the vertical plates 73 is fixed to the two opposite ends of the side baffles 72 on this side.

The vertical plates 73 are disposed on the side baffles 72 and extend towards one side closing to the detection circuit board 22, such that the connecting plate 71, two side baffles 72 and two vertical plates 73 are separately located in five different directions of the sensitive element 221 and form the total-shadow shielding zone 801 for covering the sensitive element 221, thus better protecting the sensitive element 221 from being affected by the irradiation ray.

Alternatively, the shielding frame in the above embodiment specifically may be a component in the detection circuit board, such as a battery, an electromagnetic fastener and other components that can meet the function of shielding the irradiation ray.

Alternatively, the material of the shielding frame in the above embodiment is the same as that of the shielding block 80, both of which can achieve the function of blocking the irradiation ray.

It can be understood that the shape of the shielding frame is not limited to the above solution, and can be set according to specific demands, for example, the section of the shielding frame may also be a curved surface, a polygon and the like as long as the total-shadow shielding zone formed by the shielding frame can wrap the sensitive element.

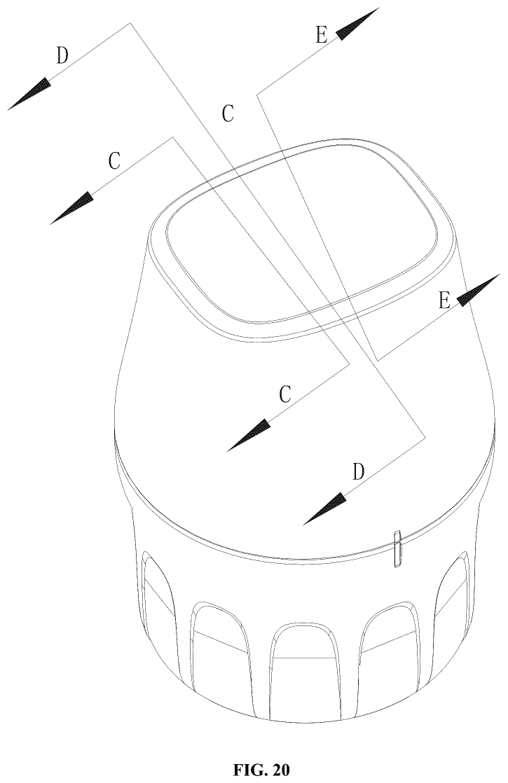

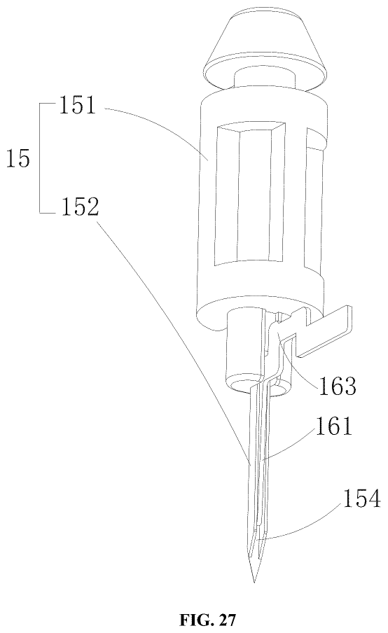

Please combine with FIGS. 20-27, embodiments of the present disclosure further provide a detector, the detector has the structure that is basically the same as that of the detector in the above embodiments, where the components with the structures that are basically the same adopt the same number, the above components are not repeatedly described any more, and the key part of the detector provided by this embodiment and the part different from other embodiments are mainly described below.

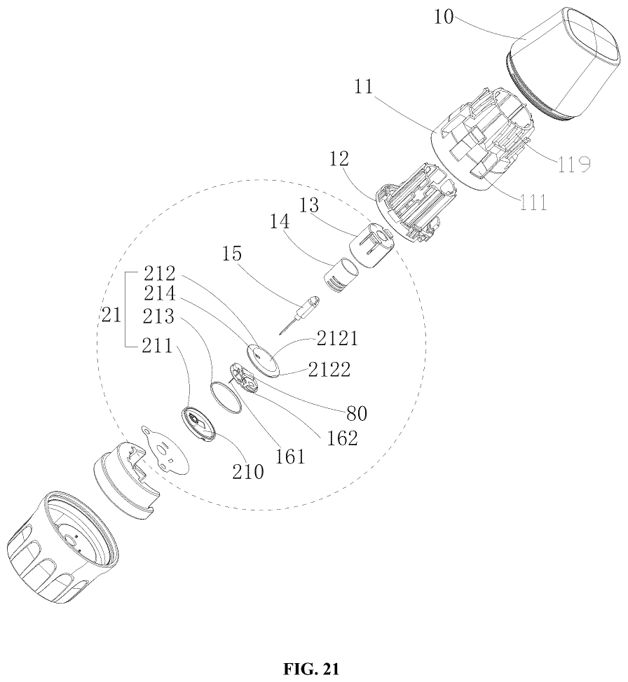

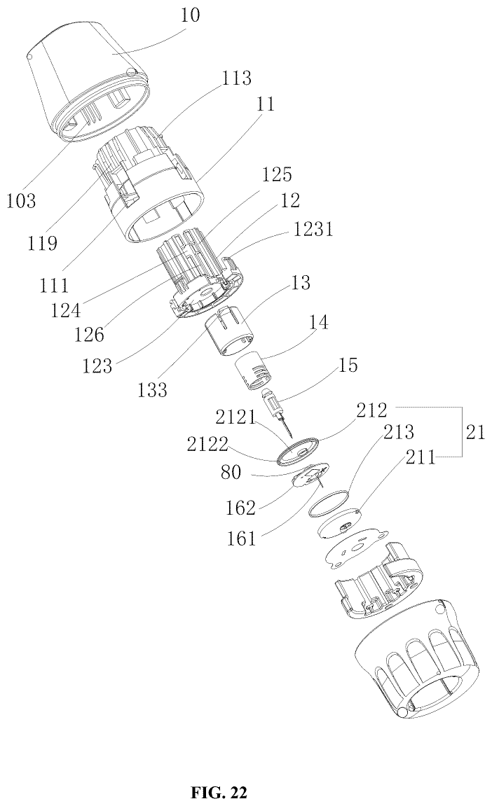

Specifically, please refer to FIG. 21 and FIG. 22, in this embodiment, the first housing 21 includes a bearing shell 211, a cover body 212 and a sealing element 213, the bearing shell 211 is hermetically connected with the cover body 212 through the sealing element 213 and forms a sealed storage cavity 210, the detection circuit board 22 is located in the storage cavity 210, and it can be understood that the sealing area of the total-shadow shielding zone includes the storage cavity 210. Hermetically arranging the detection circuit board 22 in the first housing 21 as a whole can more effectively reduce the pollution situation possibly generated when the detection circuit board 22 is not sterilized, to ensure the safety and accuracy of the detection.

Further, the first housing 21 has a cut-through hole 214, one end of the probe 161 is located in the cut-through hole 214 and stretches into the first housing 21 by passing through a connector 163 of a hole wall of the cut-through hole 214 to be electrically connected with the detection circuit board 162, the other end of the probe 161 stretches out of the cut-through hole 214 and extends towards a direction away from the pressing portion, the guide needle 15 includes a connecting rod 151 and a needle body 152 connected with the connecting rod 151, the connecting rod 151 is connected with the support bracket 13, and the needle body 152 is provided with a through hole that accommodates the probe 161 and is slidingly connected with the probe 161.

It can be understood that, in other embodiments, at least one of the bearing shell 211 and the cover body 212 may be made of a flexible material, such as silica gel, and at this time, the sealing element 213 may be omitted, that is, the bearing shell 211 and the cover body 212 can be directly and hermetically connected.

Further, the bearing shell 211 may include a bearing plate 2111 and a side wall structure 2112 connected with the bearing plate 2111, the side wall structure 2112 is provided with a sealing slot 2113, at least part of the sealing element 213 is located in the sealing slot 2113, the cover body 212 includes a main cover body 2121 and a raised structure 2122 that is connected with the main cover body 2121 and extends towards one side of the bearing plate 2111, the raised structure 2122 is used for resisting and connecting the sealing element 213, and the sealing element 213 may include a sealant, a sealing rubber ring or a combination of the sealant and the sealing rubber ring.

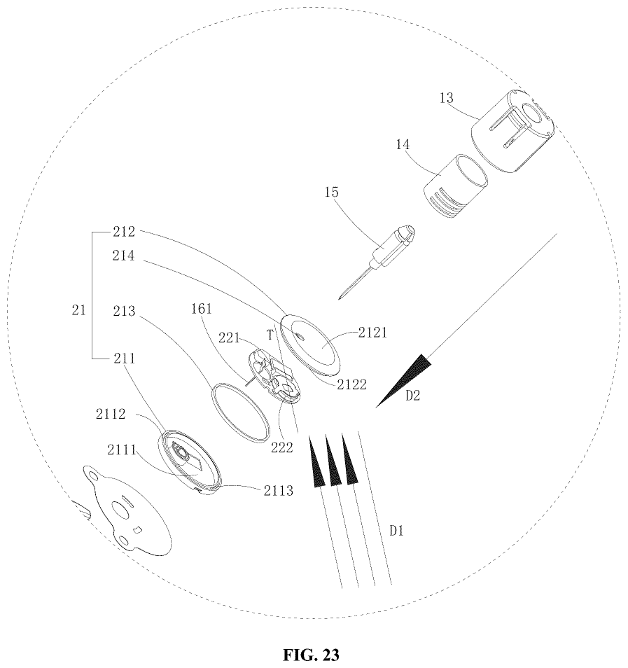

More further, please refer to FIG. 23, the shielding assembly is disposed on the detection circuit board 22, the shielding assembly and the sensitive element 221 are arranged on a path T that an irradiation source for sterilization is emitted to the detector, such that the shielding assembly can block a radiation of the irradiation source to be emitted to the sensitive element 221, and an irradiation direction D1 of the radiation is different from a pressing direction D2 of the pressing portion, and specifically, the irradiation direction D1 of the radiation may be perpendicular to the pressing direction D2 of the pressing portion.

The detection circuit board 22 is also provided with a battery module 222, the battery module 222, the shielding assembly and the sensitive element 221 are all disposed on the path T that the irradiation source for sterilization is emitted to the detector, such that the battery module 222 and shielding assembly block the radiation of the irradiation source to be emitted to the sensitive element together. It can be understood that, in other embodiments, the battery module 222 may also be replaced with other electronic devices, such as inductance, capacitance, resistance, chips or other devices.

Further, as shown in FIGS. 20-27, the detector includes a housing 10, a first barrel-shaped bracket 11, a second barrel-shaped bracket 12, a support bracket 13, an elastic element 14, a guide needle 15 and a detection assembly;
the first barrel-shaped bracket 11 is slidingly located in the housing 10 and partially located in the housing 10;
the second barrel-shaped bracket 12 is slidingly located in the first barrel-shaped bracket 11 and connected with the housing 10 through an extending connecting portion 1231, and a first through hole 124 is provided on the second barrel-shaped bracket 12;
the support bracket 13 is located in the second barrel-shaped bracket 12, and an elastic resisting portion 133 extends outside the support bracket 13;
the elastic element 14 is compressed between the second barrel-shaped bracket and the support bracket 13, such that the elastic resisting portion 133 abuts against the hole wall of the first through hole 124;
the guide needle 15 is connected with the support bracket 13, an accommodating slot 154 is provided on the guide needle 15, and the guide needle 15 is used for piercing into the sampling part;
the detection assembly is connected with one side of the first base plate 123 that is away from a second base plate of the support bracket 13, and the detection assembly includes a probe 161 that is partially accommodated in the accommodating slot 154; and
the housing 10 is used for driving the second barrel-shaped bracket to move in relative to the first barrel-shaped bracket 11, when the second barrel-shaped bracket moves to the first preset position, the guide needle 15 is pierced into the sampling part, and when moving to the second preset position, the elastic resisting portion 133 shrinks inward, such that the elastic element 14 drives the guide needle 15 to leave the sampling part.

The detection assembly can be used for detecting various data of the human body, including but not being limited to blood glucose indexes, hemoglobin, white blood cell count, blood platelet count and the like, and the following embodiments take the detection for blood glucose indexes as an example.

In the embodiments of the present disclosure, the elastic element 14 may be a spring. Since the elastic element 14 is compressed between the second barrel-shaped bracket the support bracket 13, that is, the elastic element 14 will apply an elastic force to the second barrel-shaped bracket and the support bracket 13 to enable the second barrel-shaped bracket to be away from the support bracket 13, but the elastic resisting portion 133 abuts against the hole wall of the first through hole 124, such that the elastic force generated by the elastic element 14 is applied on the hole wall of the first through hole 124 through the elastic resisting portion 133, the second barrel-shaped bracket and the support bracket 13 are also stationary relatively, and the elastic element 14 keeps a state of being compressed.

When the user adopts the detector for detecting the blood glucose, the user holds the housing 10 and aligns the detection assembly with the sampling part, the first barrel-shaped bracket 11 abuts against a vicinity of the sampling portion, and at this time the guide needle 15 is not in contact with the sampling part. Then the housing 10 is pushed to move towards one side closing to the sampling part; since the housing 10 is connected with the second barrel-shaped bracket, the support bracket 13 also abuts against the second barrel-shaped bracket through the elastic resisting portion 133, the housing 10 can drive the support bracket 13, guide needle 15 and detection assembly to move towards one side closing to the sampling part when driving the second barrel-shaped bracket to move. In a process that the second barrel-shaped bracket drives the guide needle 15 to move, the guide needle 15 is close to the sampling part gradually until the second barrel-shaped bracket moves to the first preset position, and the guide needle 15 is pierced into the sampling part. Specifically, due to insufficient hardness of the probe 161, the probe 161 is accommodated in the accommodating slot 154, and the probe 161 is brought into the sampling part by piercing the detection assembly into the sampling part, thus sampling through the probe 161 and detecting the blood glucose indexes.

The user continues to push the housing 10 to move towards one side closing to the sampling part until the second barrel-shaped bracket reaches the second preset position. At this time, the elastic resisting portion 133 moves towards one side of a first guide wall 126 that is away from the housing 10, that is, the elastic resisting portion 133 shrinks inward, such that the elastic resisting portion 133 moves out of the first through hole 124. The elastic resisting portion 133 does not abut against the hole wall of the first through hole 124 any more, the support bracket 13 is also not limited by the second barrel-shaped bracket any more, the elastic force generated by the compressed spring will push the support bracket 13 towards one side away from the first base plate 123, thus driving the guide needle 15 to move towards one side away from the sampling part, enabling the guide needle 15 to leave a to-be-sampled part, and quickly pulling out the guide needle 15 from the sampling part. Since the detection assembly is not connected with the support bracket 13, the probe 161 of the detection assembly will not be pulled out of the sampling part together with the guide needle 15, instead of remaining in the sampling part for continuous sampling detection, thus dynamically feeding back a detection result.

Through the above embodiments, the user may push the housing 10, the probe 161 is pierced into the sampling part through the guide needle 15, the support bracket 13 is not limited by the second barrel-shaped bracket any more after the guide needle 15 brings the probe 161 into the sampling part for a certain depth, the spring is released such that the guide needle 15 is quickly pulled out of the sampling part along the piercing path, so situations of strong pain, skin injury and the like of to-be-detected personnel, caused by deviating from the trajectory, relatively slow needle withdrawal and the like, do not appear easily, thus improving the experience of the to-be-detected personnel.

In one alternative embodiment, the specific structure of the detector is that the first barrel-shaped bracket 11 is partially located in the housing 10. The first barrel-shaped bracket 11 forms a cylinder of which two ends are cut-through, and the cylinder has a first cavity; and a travel hole 111 is provided on an outer wall of the first barrel-shaped bracket 11, and the travel hole 111 may be specifically a strip hole; and
the second barrel-shaped bracket includes a first base plate 123 and a first guide wall 126, the first base plate 123 is provided with a connecting portion 1231, the connecting portion 1231 passes through the travel hole 111 and is connected with the housing 10, and the connecting portion 1231 can move along a length direction of the travel hole 111; and the travel hole 111 may limit the moving direction and maximum distance of the connecting portion 1231. The first guide wall 126 is perpendicular to one side of the first base plate 123 and encloses a second cavity, and the first through hole 124 is provided on the first guide wall 126; and
the support bracket 13 is located in the second cavity.

On the one hand, the first guide wall 126 can limit the position of the support bracket 13, allowing the support bracket 13 not to generate a horizontal displacement easily in relative to the second barrel-shaped bracket; and on the other hand, the piercing direction of the guide needle 15 is enabled to be consistent with the pull-out direction thereof, to avoid injuries on the to-be-detected user due to inconsistent piercing and pull-out directions.

In one alternative embodiment, a support wall is provided on the first barrel-shaped bracket 11, and the support wall extends towards one side of the sampling part and is used for abutting against the sampling part.

The support wall is used for abutting against the sampling part, and a length of the support wall is greater than a distance from the guide needle 15 to the sampling part, such that the travel space of the guide needle 15 is reserved by the support wall abutting against the sampling part.

In one alternative embodiment, a first guide portion 113 is provided on the first barrel-shaped bracket 11, the first guide portion 113 is partially located in the first through hole 124, and when the detection assembly moves to the second preset position, the first guide portion 113 extrudes the elastic resisting portion 133 from the first through hole 124.

In a process that the second barrel-shaped bracket is driven to move closely to the sampling part along the length direction of the travel hole 111 when the user pushes the housing 10, the elastic resisting portion 133 approaches to the first guide portion 113 gradually. The elastic resisting portion 133 and the first guide portion 113 are relatively provided with two inclined surfaces, and in a process when the detection assembly moves to the second preset position, the inclined surface of the elastic resisting portion 133 is in contact with that of a first resisting portion, the elastic resisting portion 133 shrinks inward along the inclined surface of the first guide portion 113, such that the first guide portion 113 extrudes the elastic resisting portion 133 from the first through hole 124 along the inclined surface of the first guide portion 113. The second barrel-shaped bracket does not limit the elastic resisting portion 133 any more, and the elastic element 14 is released such that the support bracket 13 moves towards one side away from the sampling part, thus pulling the guide needle 15 out of the sampling part.

In one alternative embodiment, a second through hole (not shown in the drawing) is provided on the first base plate 123, and the guide needle 15 includes:
a connecting rod 151, where the connecting rod 151 penetrates through the second through hole and includes a first end and a second end that are opposite to each other, and the first end is fixed to the support bracket 13;
a needle body 152, where the needle body 152 is connected with the second end of the connecting rod 151, an accommodating slot 154 is provided on the needle body 152, and please refer to FIG. 23 for details.

The support bracket 13 drives the connecting rod 151 to move, thus driving the needle body 152 to move, thereby enabling the needle body 152 to be pierced into or pulled out of the sampling part. Since the probe 161 is accommodated in the accommodating slot 154, the probe 161 also enters the sampling part together when the needle body 152 is pierced into the sampling part.

Further, the detection assembly includes:
a detection circuit board 162, where the detection circuit board 162 is connected with one side of the first base plate 123 that is away from the support bracket 13, and used for connecting with the probe 161.

The detection circuit board 162 is also used for receiving the information detected after the probe 161 enters the sampling part. The detection circuit board 162 may also transmit the information received to other terminals, so as to inform the users of the specific information detected in various forms (such as characters and charts).

In one alternative embodiment, an inner wall of the housing 10 is provided with an arc-shaped bulge 101, and the arc-shaped bulge 101 is located on one side of the first base plate 123 that faces the first guide wall 126; and
an outer wall of the first barrel-shaped bracket 11 is provided with a first limiting portion 117, and the first limiting portion 117 is located between the arc-shaped bulge 101 and the first base plate 123 and abuts against the arc-shaped bulge 101.

The user operates the housing 10 to move towards one side closing to the sampling part, and before the second barrel-shaped bracket moves to the second preset position, the first limiting portion 117 will abut against the arc-shaped bulge 101. After contacting with the arc-shaped bulge 101, the user applies a relatively great thrust to the housing 10 since the first limiting portion 117 has a certain elasticity, such that the first limiting portion 117 exceeds the arc-shaped bulge 101 gradually. However, the speed that the user pushes the housing 10 becomes slow due to the interference on the first limiting portion 117 by the arc-shaped bulge 101, after the first limiting portion 117 moves to the highest point of the arc-shaped bulge 101, the first barrel-shaped bracket 11 is not interfered by the first limiting portion 117 any more, the user will keep a relatively great thrust due to inertia, such that the housing 10 and the whole second barrel-shaped bracket have a relatively great accelerated speed to move towards one side of the sampling part at a moment that the first limiting portion 117 exceeds the highest point of the arc-shaped bulge 101, thus quickly piercing a needling component into the sampling part. When the user has not yet felt the pain of piercing into the sampling part completely, the guide needle 15 has been pierced into the sampling part, to avoid generating a great pain when slowing down the piercing process due to the user's pain.

In one alternative embodiment, a first guide slot 125 is provided on the outer wall of the second barrel-shaped bracket, and a second guide portion (not shown in the drawings) corresponding to the first guide slot 125 is provided on the inner wall of the first barrel-shaped bracket 11; and
the first guide slot 125 is used for limiting the moving direction of the second guide portion.

When the user controls the movement of the housing 10, the housing 10 drives the second barrel-shaped bracket to move, the second guide portion moves along the direction of the first guide slot 125, thus limiting the movement of the housing 10 and the second barrel-shaped bracket, and avoiding the deviation generated by unstable movement due to improper user operations.

In one alternative embodiment, a second guide slot 119 is provided on the outer wall of the first barrel-shaped bracket 11, and a third guide portion 103 corresponding to the second guide slot 119 is provided on the inner wall of the housing 10; and
the second guide slot 119 is used for limiting the moving direction of the third guide portion 103.

The cooperative effect of the second guide slot 119 and the third guide portion 103 is basically the same as that of the first guide slot 125 and the second guide portion, both of which are used for avoiding the deviation generated by unstable movement due to improper user operations, and repetitions are not made here.

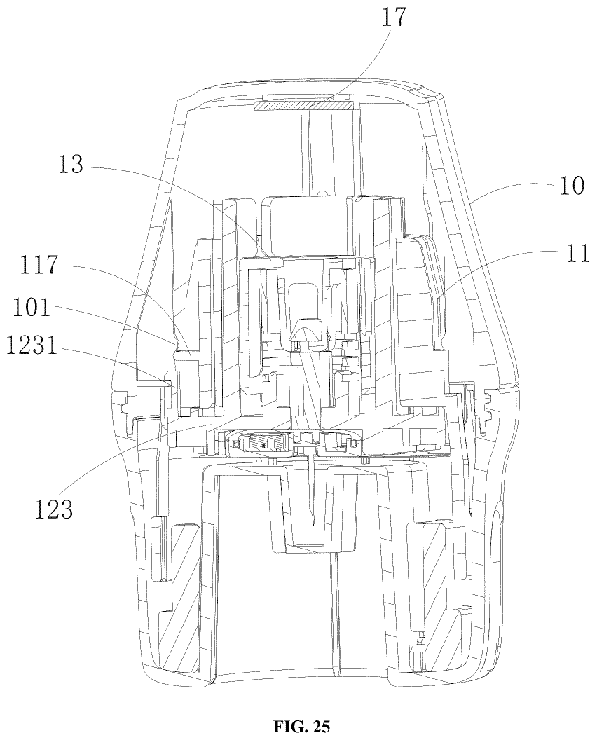

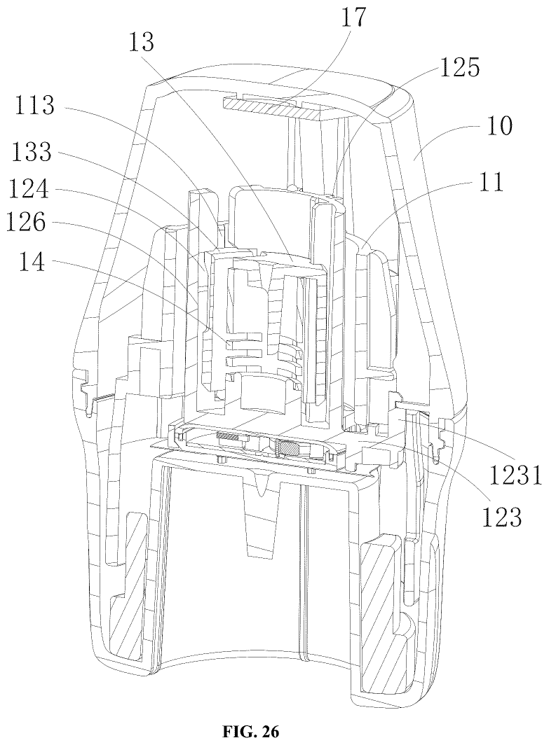

In one alternative embodiment, please refer to FIG. 25 and FIG. 26, the detector further includes a shock absorbing assembly 17, and the shock absorbing assembly 17 is located on one side of the support bracket 13 that is away from the first base plate 123 and fixed to the inner wall of the housing 10.

The elastic force, generated when the elastic element 14 is released, will push the support bracket 13 towards one side, and the support bracket 13 will collide with the housing 10 to generate a vibration, which may cause the guide needle 15 to generate the deviation. However, the shock absorbing assembly 17 can replace the housing 10 to bear the shock generated by the support bracket 13, and weaken the vibration brought by the shock, thus avoiding the deviation of the guide needle 15 caused by the vibration. Specifically, the shock absorbing assembly 17 may be a sponge mat.

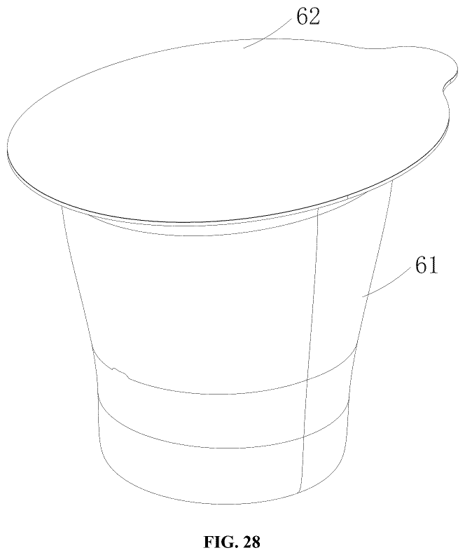

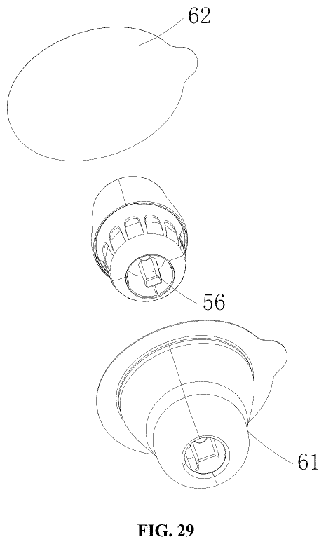

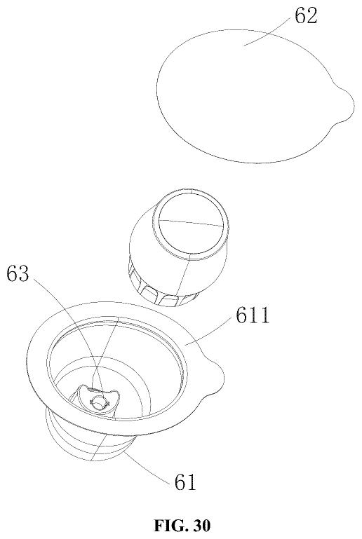

Please refer to FIGS. 28-30, embodiments of the present disclosure further provide a detector assembly, the detector assembly may adopt the detector in any one of the above embodiments and the outer package for sealing and packaging the detector, the outer package may include a main housing 61 and an easy-to-tear film 62, the main housing 61 is provided with an accommodating space and an opening communicating with the accommodating space, the main housing 61 has an end face 611 surrounding the opening, an outer contour of the easy-to-tear film 62 is basically consistent with that of the end face 611, and the easy-to-tear film 62 is attached to the end face 611 through colloid; and the main housing 61 is also provided with a first counterpoint structure 63, the bottom of the detector (such as on the second outer shell 52) may have a second counterpoint structure 56, and the second counterpoint structure 56 is in counterpoint fit with the first counterpoint structure 63, such that the detector may be disposed in the main housing 61 according to a preset orientation.

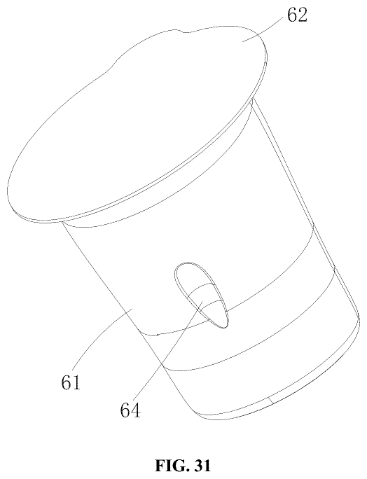

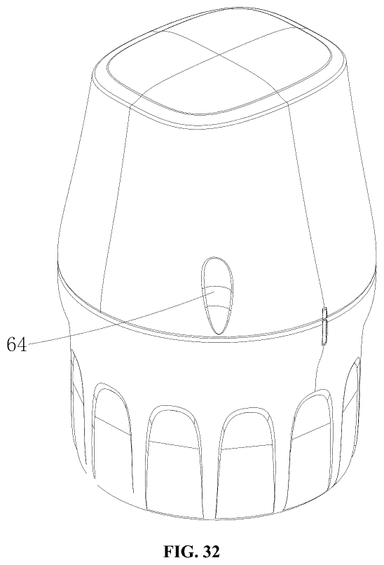

Further, please refer to FIG. 31, in one alternative embodiment, the main housing 61 may also be provided with a third counterpoint structure 64. The third counterpoint structure 64 is used for counterpoint fit with a fourth counterpoint structure 94 on the bearing frame 91 of the sterilization system, such that the orientation of the detector may be fixed, facilitating the sterilization for the detector by using the irradiation ray in the preset direction. The third counterpoint structure 64 may be a counterpoint groove. The fourth counterpoint structure 94 may be a counterpoint bulge. It can be understood that, as shown in FIG. 32, in another alternative embodiment, when the detector is not provided with the outer package, the third counterpoint structure 64 may be disposed on the outer surface (such as the outermost housing) of the detector.

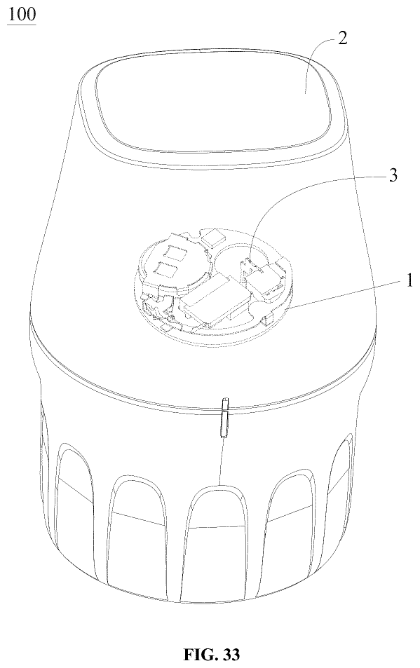

Referring to FIG. 33, FIG. 33 is a schematic diagram of a partial structure of a detector according to a fourteenth embodiment of the present disclosure. It can be understood that other parts (not shown) such as a guide needle 4, a bracket, an elastic element, a pressing element, and a sealing element are further arranged in the detector 100. Certainly, the detector 100 can be specifically designed according to a specific application scenario, and may not include the foregoing parts not shown. As other parts not shown in the accompanying drawings are not the key innovation technology points of the present disclosure, these parts will not be described in detail and will not be shown in the accompanying drawings in this embodiment.

The detector 100 may be a blood glucose detector configured to measure a blood glucose level of a user or a patient.

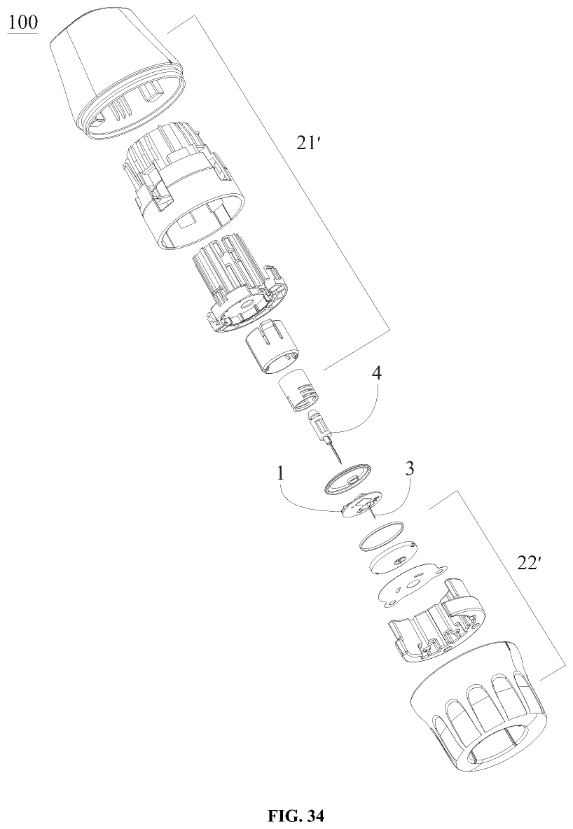

Specifically, referring to FIG. 34, FIG. 34 is a three-dimensional exploded view of the detector shown in FIG. 33. In this embodiment, the detector 100 includes a housing 2, and a control module 1, a sensing unit 3, and a guide needle 4 which are arranged inside the housing 2. The guide needle 4 is configured to: pierce a sampling site to obtain a sample, and obtain raw data from the sample through the sensing unit 3 or transmit the sample to the control module 1 for processing, conversion, and transmission. In this embodiment, the housing 2 includes an upper housing assembly 21′ and a lower housing assembly 22′. The upper housing assembly 21′ and the lower housing assembly 22′ are enclosed to form a cavity. The control module 1, the sensing unit 3, and the guide needle 4 are all arranged inside the cavity. Since the housing 2 is not an innovative design point of this embodiment of the present disclosure, it will not be elaborated here. It can be understood that the sensing unit 3 may be equivalent to a probe the above implementation I to implementation XI.

The detector 100 may also be a continuous detector 100, which is worn on a specific body part of a user or a patient, for continuous monitoring, data analysis, and management.

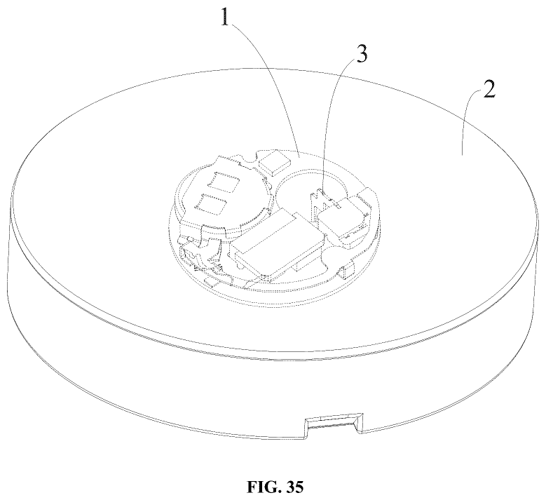

Specifically, referring to FIG. 35, FIG. 35 is a schematic diagram of a partial structure of another detector according to a fourteenth embodiment of the present disclosure. In this embodiment, the detector 100 may not include the guide needle 4. The detector 100 can be directly worn on a body part of the user or the patient to continuously monitor blood glucose of the user or the patient, and transmit monitored data to the control module 1 for processing, conversion, and transmission through the sensing unit 3. In this embodiment, the detector 100 includes a housing 2 and a control module 1 arranged inside the housing 2. The control module 1 processes, converts, and transmits acquired raw data. The detector 100 may further include a sensing unit 3. The sensing unit 3 is configured to: obtain relevant raw data of the blood glucose and transmit the raw data to the control module 1 for processing, conversion, and transmission.

The control module 1 in the foregoing embodiment may be connected to an external electronic device such as a mobile phone, a tablet, and a computer, and process, convert, and transmit the blood glucose detected data to external device, thereby facilitating subsequent big data analysis and organization, and facilitating blood glucose level management for the user.

Based on FIG. 33 and FIG. 35, it can be seen that the specific structure and appearance of the housing 2 of the detector 100 are designed according to a product design requirement. The structure of the housing 2 shown in FIG. 33 and FIG. 35 is only a schematic diagram for ease of understanding.

Due to different application scenarios and different internal structures of the detector 100, other parts are not the key innovative technology points of this embodiment of the present disclosure. Therefore, the following embodiments of the present disclosure will provide a detailed description for the key innovative part, namely, the control module 1, in conjunction with the accompanying drawings.

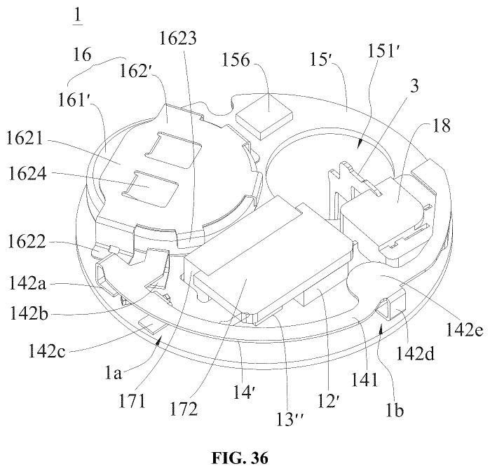

Referring to FIG. 36, FIG. 36 is a three-dimensional diagram of a control module of a detector according to an embodiment of the present disclosure. The control module 1, as a part of the detector 100, includes a circuit board. The circuit board is specifically a detection circuit board 15′. The other components of the control module 1 are all arranged on the detection circuit board 15′. The detection circuit board 15′ has a first mounting site 151′. The first mounting site 151′ is configured to arrange the above sensing unit 3, and the first mounting site 151′ is a hole that penetrates through the detection circuit board 15′. Specifically, the first mounting site 151′ includes a head mounting region 1511 for mounting part of the sensing unit 3 in a penetrating manner, and a tail mounting region 1512 communicated to the head mounting region 1511. The head mounting region 1511 is a circular hole. The tail mounting region 1512 is rough a long-strip hole. A diameter of the head mounting region 1511 is greater than 2.5 mm. The tail mounting region 1512 has a length greater than 2.5 mm and a width greater than 1 mm. This setting can ensure miniaturization of the size of the first mounting site 151′ and can mount the sensing unit 3. Referring to FIG. 36 to FIG. 38, the control module 1 includes a sensing unit connector 18. The sensing unit connector 18 is arranged on the detection circuit board 15′. Specifically, the sensing unit connector 18 is arranged in the tail mounting region 1512. The sensing unit connector 18 is configured to be electrically connected between the sensing unit 3 and the main control unit 12′.

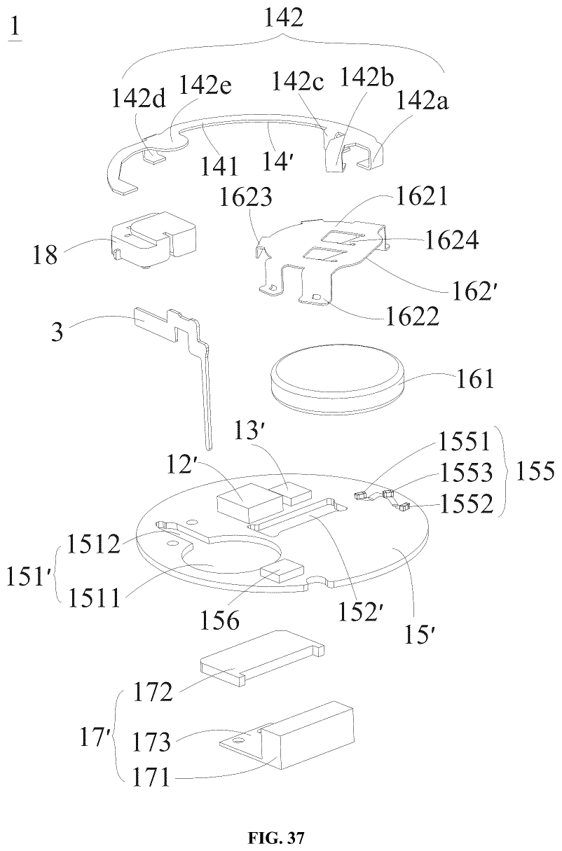

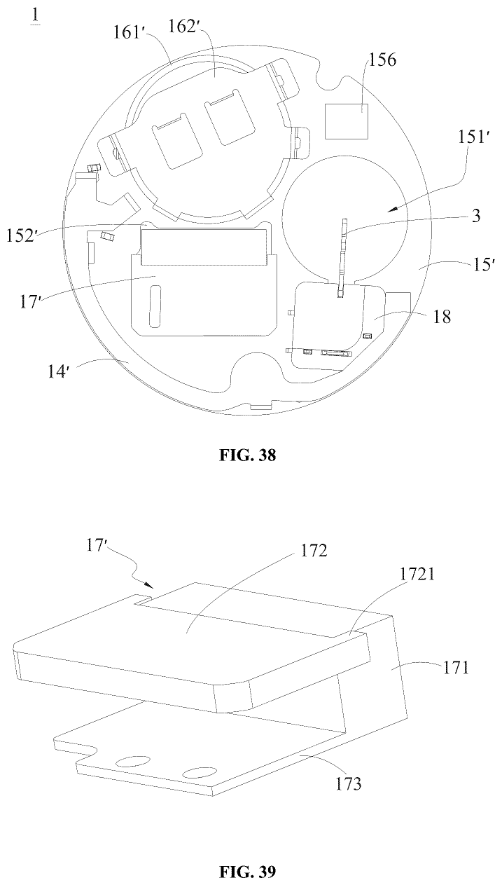

Specifically, referring to FIG. 36, FIG. 37, and FIG. 38, the control module 1 includes a first antenna 14′, a main control unit 12′, a wireless communication control unit 13, a second antenna 11′, and a shielding assembly 17′. The second antenna 11′, the main control unit 12′, the wireless communication control unit 13, the first antenna 14′, and the shielding assembly 17′ are all arranged on the detection circuit board 15′.

The wireless communication control unit 13 is electrically connected to the main control unit 12′, and the wireless communication control unit 13 is configured to: receive detected data and output a first radio frequency signal. The first antenna 14′ is electrically connected to the wireless communication control unit 13. The first antenna 14′ is configured to receive the first radio frequency signal for wireless transmission. The second antenna 11′ is configured to receive the second radio frequency signal. The second radio frequency signal is an NFC radio frequency signal. The main control unit 12′ is electrically connected to the second antenna 11′. The main control unit 12′ is configured to: receive the NFC radio frequency signal to start working, and after starting working, process raw data output by the sensing unit 3 to obtain the detected data.

Referring to FIG. 39, in this embodiment, the shielding assembly 17′ includes a side plate 171 connected to the detection circuit board 15′, a top plate 172 connected to the side plate 171, and a base plate 173 connected to one end of the side plate 171 away from the top plate 172. The side plate 171 is adjacent to the main control unit 12′, and the top plate 172 is located on one side of the main control unit 12′ away from the detection circuit board 15′. The base plate 173 and the top plate 172 are respectively arranged on the same side of two ends of the side plate 171 to form a concave structure. The base plate 173 is located on one side of the detection circuit board 15′ away from the top plate 172, so that the top plate 172 and the base plate 173 are respectively located on two sides of the detection circuit board 15′. The shielding assembly 17′ forms a full shadow shielding region for protecting the main control unit 12′, to block at least part of an irradiation ray. In this embodiment, the top plate 172 is located directly above the main control unit 12′. The base plate 173 is located directly below the main control unit 12′. The side plate 171 is located on one side of the main control unit 12′, thereby surrounding the main control unit 12′. This ensures that during irradiation sterilization of the irradiation ray, the side plate 171, the top plate 172, and the base plate 173 block a path of the irradiation ray, thus forming the full shadow shielding region to protect the main control unit 12′ therein, and avoiding the irradiation of the irradiation ray. A material of the shielding assembly 17′ includes but is not limited to lead, stainless steel, tungsten, or other high-density polymers.

Specifically, in this embodiment, the top plate 172 is provided with a connecting portion 1721. The connecting portion 1721 is connected to one end of the side plate 171 away from the base plate 173. The base plate 173 is integrally formed with the side plate 171. The connecting portion 1721 and the side plate 171 are connected in a welded, crimped, or adhered manner. In this way, the side plate 171 can pass through the detection circuit board for 15′, and is then connected to the top plate 172 to facilitate production and assembling of the product.

In a changed embodiment, the top plate and the side plate can be integrally formed, mounted on the detection circuit board, and connected to the base plate.

In other embodiments, the top plate and the base plate may be connected to two ends of the side plate. After the side plate is mounted on the detection circuit board, the base plate and the top plate are respectively connected to two ends of the side plate.

In another embodiment, the top plate, the base plate, and the side plate are respectively arranged on the detection circuit board, or the side plate is connected to one of the top plate or base plate, and the other one is separately arranged on the detection circuit board as long as the side plate, the top plate, and the base plate are located on three side surfaces of the main control unit to block the irradiation path of the irradiation ray and form the full shadow shielding region to protect the main control unit.

Specifically, referring to FIG. 36 to FIG. 38, the detection circuit board 15′ further has a second mounting site 152′. The shielding assembly 17′ is arranged at the second mounting site 152′. Specifically, the side plate 171 is arranged at the second mounting site 152′ in a penetrating manner. The second mounting site 152′ is a hole that penetrates through the detection circuit board 15′. Specifically, the second mounting site 152′ is a roughly rectangular hole with a length greater than or equal to 3 mm and a width greater than or equal to 1 mm. Roughly trapezoidal extension open pores are designed at four corner positions of the rectangular hole of the second mounting site 152′, and distances between the roughly trapezoidal extension open pores in a length direction and width direction of the second mounting site 152′ are greater than 0.15 mm.

Referring to FIG. 39, in this embodiment, the top plate 172 has a length greater than 3.2 mm, a width greater than 2.5 mm, and a thickness greater than 0.3 mm. The side plate 171 has a width greater than 0.5 mm and a height greater than 2 mm. The base plate 173 has a length greater than 3 mm, a width greater than 2 mm, and a thickness greater than 0.15 mm. To ensure that the full shadow shielding region can completely shield and protect the main control unit 12′, a size of an orthographic projection region of the shielding assembly 17′ on the detection circuit board 15′ is as follows: The length is greater than 3 mm and the width is greater than 3 mm. This design ensures that the shielding assembly 17′ meets the requirements for shielding the irradiation ray while being miniaturized.

Compared with the existing technology, the control module 1 provided in the present disclosure achieves efficient data processing, wireless communication, and data transmission by connection and cooperation of the main control unit 12′, the wireless communication control unit 13, and the first antenna 14′ on the detection circuit board 15′, which facilitates analysis and management by using big data. Furthermore, the shielding assembly 17′ is arranged around the three side surfaces of the main control unit 12′ to form the full shadow shielding region to protect the main control unit 12′, thus blocking the irradiation ray. This can ensure all-round shielding and blocking of the irradiation direction of the irradiation ray, thereby achieving all-round protection for sensitive components during irradiation sterilization. The base plate 173 is integrally formed with the side plate 171 and then connected to the top plate 172, which facilitates the side plate 171 to pass through the detection circuit board 15′ and facilitates the mounting of the shielding assembly 17′. A mounting sequence is that the side plate 171 and the base plate 173 are mounted first, and then the top plate 172 is mounted, which facilitates the production and the assembling and improves the production efficiency. In addition, the second antenna 11′ is configured to receive the NFC radio frequency signal. The main control unit 12′ is electrically connected to the second antenna 11′. The main control unit 12′ only starts working after receiving the NFC radio frequency signal. When an external interactive device transmits the NFC radio frequency signal to the second antenna 11′, the main control unit 12′ receives the NFC radio frequency signal and starts working. On the contrary, when the external interactive device does not transmit the NFC radio frequency signal to the second antenna 11′, the main control unit 12′ is in a standby state, thus achieving extremely low overall power consumption.

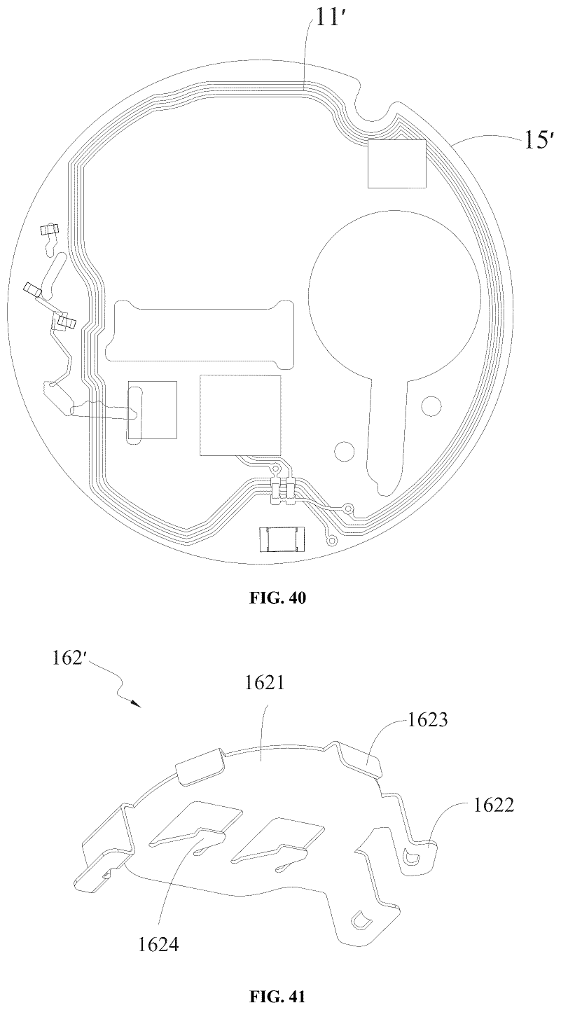

Referring to FIGS. 36 to 38 and FIG. 40, the second antenna 11′ includes a conductive line formed on the detection circuit board 15′. The second antenna 11′ is formed on a bottom layer and sub-bottom layer of the detection circuit board 15′ through a winding layout. A number of turns of the second antenna 11′ is greater than or equal to four. This makes the second antenna 11′ arranged widely, facilitating the receiving of the second radio frequency signal. For example, the detection circuit board 15′ may include a first base layer, a second base layer, a third base layer, and a fourth base layer which are stacked in sequence. The first base layer is not provided with a second antenna 11′. The second base layer is not provided with a second antenna 11′. The third base layer may be provided with two turns of second antennas 11′. The fourth base layer is provided with two turns of second antennas 11′. The detection circuit board 15′ further includes a via hole. The via hole penetrates through the first base layer, the second base layer, the third base layer, and the fourth base layer. The second antennas 11′ in different base layers are connected through the via hole.

Referring to FIG. 36 to FIG. 38, to achieve power supplying, the control module 1 further includes a battery module 16. The battery module 16 is arranged on the detection circuit board 15′. Specifically, the battery module includes a battery 161′ and a battery holder 162′. The battery holder 162′ is connected to the detection circuit board 15′, and the battery 161′ is arranged between the battery holder 162′ and the detection circuit board 15′. A cavity is formed between the battery holder 162′ and the detection circuit board 15′, which can accommodate the battery 161′. The battery holder 162′ has an opening in one side. A width of the opening is greater than 7.8 mm, to mount the battery 161′ into the cavity. A material of the battery holder 162′ is stainless steel.

Referring to FIG. 41, to ensure that the battery 161′ is firmly mounted between the battery holder 162′ and the detection circuit board 15′, the battery holder 162′ includes a main body portion 1621 and at least two welding legs 1622, at least one blocking leg 1623, and at least one push spring piece 1624 which are arranged on the main body portion 1621. The main body portion 1621 is arranged on one side of the battery 161′ away from the detection circuit board 15′. The at least two welding legs 1622 are opposite to each other and are connected to the detection circuit board 15′. The blocking leg 1623 collides with the battery 161′ to limit the battery 161′ between the battery holder 162′ and the detection circuit board 15′. The push spring piece 1624 collides with the battery 161′ to press the battery 161′ towards the detection circuit board 15′. A height of the battery holder 162′ is greater than 1.5 mm, and a thickness of the main body portion 1621 is greater than 0.1 mm. Specifically, in this embodiment of the present disclosure, three welding legs 1622 are included. Two welding legs 1622 are respectively arranged on two sides of the opening, and the other welding leg 1622 is arranged between the blocking leg 1623 and one of the welding legs 1622. Two blocking legs 1623 are included. One blocking foot 1623 is opposite to the opening. Two push spring pieces 1624 are included. The push spring pieces 1624 have a rigidity and an elasticity. When the battery 161′ enters the cavity, the push spring pieces 1624 are compressed to move in a direction away from the detection circuit board 15′, so that the push spring pieces 1624 have the elasticity towards the detection circuit board 15′, and then the battery 161′ is compressed to be fixed in the cavity. The two blocking legs 1623 collide with an outer side wall of the battery 161′ to prevent the battery 161′ from moving out from a side opposite to the opening. The width of the opening of the battery holder 162′ is greater than 7.8 mm to facilitate the mounting of the battery 161′. This design of and the size limitation on the battery holder 162′ can ensure that the battery holder 162′ can stably fix the battery 161′ on the detection circuit board 15′ and maintain the strength of the battery holder 162′, and can further achieve miniaturization and further reduce the size of the product.

To further avoid damage to the main control unit 12′ caused by the irradiation ray, the battery module 16, the shielding assembly 17′, and the main control unit 12′ are located on one straight line. The battery module 16 and the shielding assembly 17′ are configured to jointly block at least part of the irradiation ray, thereby ensuring the blocking of the irradiation ray in this direction. Certainly, in other embodiments, only the battery module 16 can be used to shield and block the irradiation ray.

In this embodiment, the wireless communication control unit 13 is also a sensitive unit that is easily damaged by radiation. Therefore, the wireless communication control unit 13 is arranged in the full shadow shielding region. Specifically, the wireless communication control unit 13 is arranged in parallel with the main control unit 12′ in a length direction of the second mounting site 152′. In this way, the full shadow shielding region can better avoid damage to the wireless communication control unit 13 caused by the irradiation ray, thereby ensuring the control accuracy and service life of the control module 1.

Specifically, to further reduce the volume of the control module 1 to achieve the miniaturization of the detector 100, in this embodiment, the detection circuit board 15′ is circular, and the battery module 16, the first mounting site 151′, the sensing unit connector, the main control unit 12′, and the wireless communication control unit 13 are arranged in sequence around the second mounting site 152′. This design can make the control module 1 have a more compact layout design and a smaller volume.

To further properly use a space of the detection circuit board 15′ to reduce the volume, two ends of the second antenna 11′ are respectively connected to the main control unit 12′ and form a ring-shaped region. Specifically, the battery module 16, the first mounting site 151′, the sensing unit connector 18, the main control unit 12′, the wireless communication control unit 13, and the second mounting site 152′ are all located in the ring-shaped region. This design facilitates the processing, conversion, and transmission of the detected data between the second antenna 11′ and the sensing unit 3, the main control unit 12′, as well as the wireless communication control unit 13, thus achieving efficient data processing, wireless communication, and data transmission, and facilitates analysis and management by using big data. Furthermore, the second antenna 11′, the sensing unit 3, the main control unit 12′, the wireless communication control unit 13, and the shielding assembly 17′ are arranged more compactly in spatial structure, which can further reduce the overall size of the detector 100, thereby miniaturizing the product.

The first antenna 14′ is a small-sized patch steel antenna. The first antenna 14′ is arranged around peripheries of the battery module 16, the second mounting site 152′, the wireless communication control unit 13, the main control unit 12′, and the sensing unit connector 18. This can further reduce the volume of the product and make proper use of the space of the detection circuit board 15′, thus making the layout more compact and proper. This design facilitates the processing, conversion, and transmission of the detected data between the first antenna 14′ and the sensing unit 3, the main control unit 12′, as well as the wireless communication control unit 13, thus achieving efficient data processing, wireless communication, and data transmission, and facilitates a terminal device to perform analysis and management by using big data. Furthermore, the spatial structural arrangement of the first antenna 14′, the sensing unit 3, the main control unit 12′, the wireless communication control unit 13, and the shielding assembly 17′ is more compact, which can further reduce the overall size of the detector 100, thereby miniaturizing the product.

The first antenna 14′ is arranged around a periphery of the second antenna 11′. By this design, the first antenna 14′ and the second antenna 11′ are arranged more compactly in spatial structure, which can further reduce the overall size of the detector 100, thereby miniaturizing the product.

Referring to FIG. 36 to FIG. 38 and FIG. 42, the first antenna 14′ is a steel sheet antenna. The steel sheet antenna includes an antenna main body 141 arranged above the detection circuit board 15′, and at least one feed portion 142 connected to the antenna main body 141 and the detection circuit board 15′. The at least one feed portion 142 is electrically connected to the wireless communication control unit 13 through a microstrip. In this design, the first radio frequency signal output by the wireless communication control unit 13 is transmitted to the feed portion 142 through the microstrip, and then is transmitted to the antenna main body 141 through the feed portion 142 for wireless transmission.

The microstrip includes a line formed on the detection circuit board 15′. The microstrip is a commonly used transmission line form in microwave and millimeter wave frequency bands. Its characteristic is that an electromagnetic field is mainly limited to a thin dielectric layer, and transmission and control of electromagnetic waves are achieved through three basic parts: a patch, a ground plate, and a dielectric substrate. In this embodiment, a characteristic impedance of the microstrip is 50 Ω. By implementing electrical connection through the microstrip, the layout and wiring between the units are simplified, so that the stability of the circuit is improved, the electromagnetic interference is reduced; frequency matching is optimized; and the performance of a wireless communication devices is improved.

At least one feed portion 142 is provided with an antenna clearance zone. The antenna clearance zone is a space between an antenna and its surrounding objects, which is crucial for the performance of the antenna. The antenna clearance zone ensures that the antenna has a sufficient space to avoid shielding or interference, thus ensuring an omnidirectional communication effect of the first antenna 14′. Arranging the antenna clearance zone in the feed portion 142 can ensure that a signal of the first antenna 14′ is less obstructed, thereby improving the transmission quality and coverage range of the signal of the first antenna 14′. It is worth mentioning that the second antenna 11′ and the first antenna 14′ can set a sufficient antenna clearance zone in advance on the detection circuit board 15′ for avoidance, so that the transmission efficiencies of both the second antenna 11′ and the first antenna 14′ can meet a requirement.

Specifically, referring to FIG. 36 to FIG. 38 and FIG. 42, at least one feed portion 142 includes a first pin 142a, a second pin 142b, a third pin 142c, and a fourth pin 142d. The first pin 142a and the second pin 142b are arranged at one end of the antenna main body 141. The fourth pin 142d is arranged in the middle of the antenna main body 141. The third pin 142c is arranged between the first pin 142a or the second pin 142b and the fourth pin 142d. If one end of the antenna main body 141 where the first pin 142a and the second pin 142b are located is a head, and the other end is a tail, the fourth pin 142d is arranged in a middle position between the head and tail of the antenna main body 141. The first pin 142a and the second pin 142b are both feed ground pins. The third pin 142c is an antenna feed point. The fourth pin 142d is suspended or is a feed ground pin. In this embodiment, the tail of the antenna main body 141 is suspended, so as not to interfere with the second antenna 11′.

The first pin 142a and the second pin 142b are close to the battery module 16. By this design, it is convenient for electrical connection between the first pin 142a, as well as the second pin 142b, and the battery module 16, and the first pin 142a, the second pin 142b, and the battery module 16 are arranged more compactly in spatial structure, which can further reduce the overall size of the detector 100, thereby miniaturizing the product.

The third pin 142c is close to the wireless communication control unit 13 and is electrically connected to the wireless communication control unit 13 through a microstrip. By this design, it is convenient for electrical connection between the third pin 142c and the wireless communication control unit 13, and the third pin 142c and the wireless communication control unit 13 are arranged more compactly in spatial structure, which can further reduce the overall size of the detector 100, thereby miniaturizing the product.

The fourth pin 142d is close to the main control unit 12′. By this design, it is convenient for electrical connection between the fourth pin 142d and the main control unit 12, and the fourth pin 142d and the main control unit 12 are arranged more compactly in spatial structure, which can further reduce the overall size of the detector 100, thereby miniaturizing the product.

The antenna clearance zone includes a first clearance zone la. The third pin 142c is arranged in the first clearance zone la. By this design, it can ensure that the first radio frequency signal output by the wireless communication control unit 13 is less obstructed, thereby improving the transmission quality and coverage range of the first radio frequency signal.

The antenna clearance zone further includes a second clearance zone 1b. The fourth pin 142d is arranged in the second clearance zone 1b. By this design, it can ensure that the NFC radio frequency signal received by the main control unit 12′ is less obstructed, thereby improving the transmission quality and coverage range of the NFC radio frequency signal.

More specifically, the first pin 142a, the second pin 142b, the third pin 142c, and the fourth pin 142d have the same heights. A height of the antenna main body 141 is greater than 1 mm. A thickness of the antenna main body 141 is greater than 0.1 mm. A width of the antenna main body 141 is greater than 0.8 mm. A distance between a bonding pad of each of the third pin 142c and the fourth pin 142d and a copper pavement region on the detection circuit board 15′ is greater than 0.8 mm. A length of each of the third pin 142c and the tail of the antenna main body 141 is greater than 15 mm. By properly designing the heights of the first pin 142a, the second pin 142b, the third pin 142c, and the fourth pin 142d, and the size and height of the antenna main body 141, it is easy to achieve the electrical connection between the corresponding components and connect the wireless communication control unit 13 to a terminal device such as a mobile phone through the radio frequency signal emitted by the steel sheet antenna. Blood glucose data, temperature data, and the like are uploaded to the terminal device such as the mobile phone to ensure the stability of signal transmission.

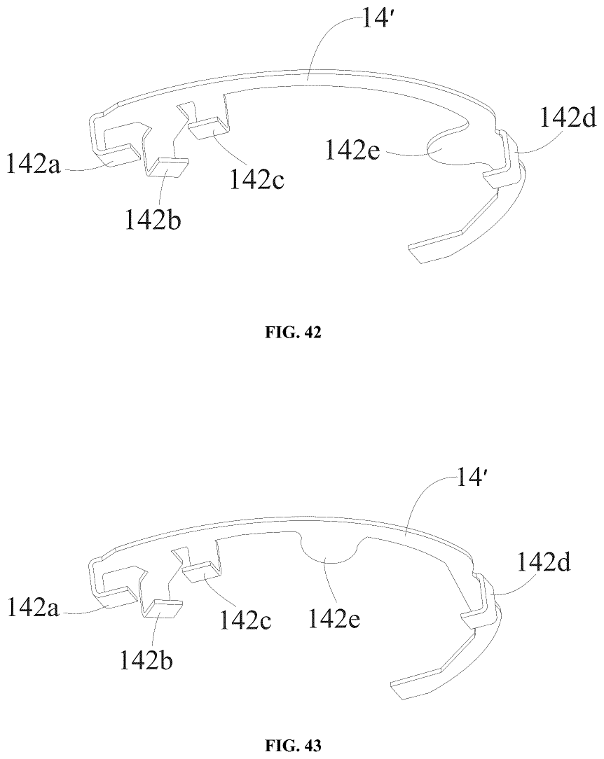

The shape and structure of the steel sheet antennas can be designed in various ways. In this embodiment, the structure of the steel sheet antenna is shown in FIG. 42. The antenna main body 141 is further provided with a nearly circular convex sheet 142e. The convex sheet 142e is connected to the fourth pin 142d and is arranged in a middle position of the antenna main body 141. The arrangement of the convex sheet 142e can transmit a signal to the terminal device such as the mobile phone more stably.

In other embodiments, the structure of the steel sheet antenna may be as shown in FIG. 43. The fourth pin 142d may be arranged at a portion, close to the tail end, of the antenna main body 141, and the convex sheet 142e and the fourth pin 142d are arranged at different positions of the antenna main body 141.

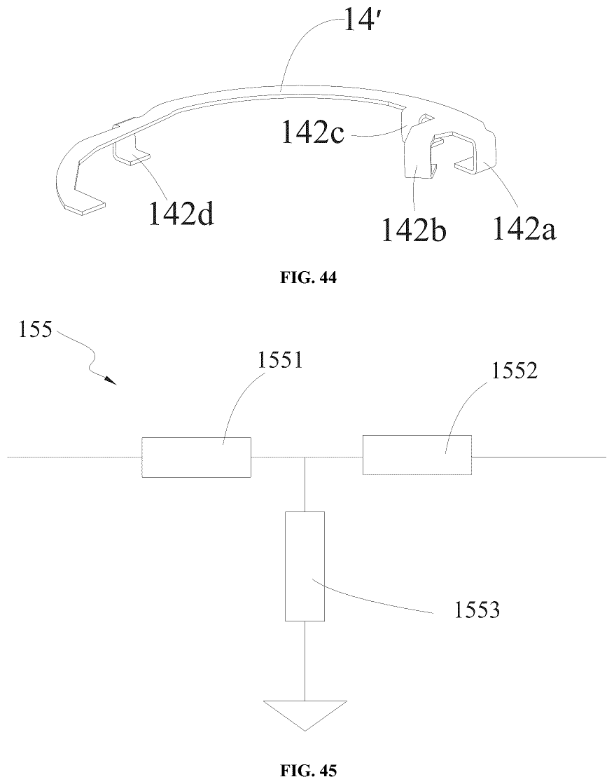

In another embodiment, the structure and shape of the steel sheet antenna may still be as shown in FIG. 44. The fourth pin 142d is close to the tail portion of the antenna main body 141, and no convex sheet 142e is arranged on the antenna main body 141.

Referring to FIG. 37 and FIG. 45, in this embodiment, the control module 1 further includes a matching network 155. The matching network 155 is electrically connected to the wireless communication control unit 13 and the steel sheet antenna via the microstrip. The matching network 155 is a T-shaped network. The matching network 155 includes a first matching unit 1551, a second matching unit 1552, and a third matching unit 1553. One of the first matching unit 1551 and the second matching unit 1552 is connected to the wireless communication control unit 13, and the other one of the first matching unit 1551 and the second matching unit 1552 is connected to the steel sheet antenna. The third matching unit 1553 is connected between a node between the first matching unit 1551 and the second matching unit 1552, and a ground. The first matching unit 1551 is an inductive or capacitive element, and the second matching unit 1552 is an inductive or capacitive element. The third matching unit 1553 is an inductive or capacitive element. Specifically, the first matching unit 1551 is a 33 pF capacitor. The second matching unit 1552 is a 0 Ω resistor. The third matching unit 1553 is not welded. By adjusting device values of the first matching unit 1551, the second matching unit 1552, and the third matching unit 1553, the matching network 155 transmits a radio frequency signal to the steel sheet antenna after reaching a matched state, thereby reducing the power consumption and ensuring the stability of radio frequency signal transmission.

Referring to FIG. 36 to FIG. 38, the control module 1 further includes a temperature sensor 156. The temperature sensor 156 is electrically connected to the main control unit 12′ or the wireless communication control unit 13 to acquire temperature information and transmit the information to the main control unit 12′ or the wireless communication control unit 13. The wireless communication control unit 13 includes a Bluetooth communication chip. The Bluetooth communication chip is a low-power BLE Bluetooth communication chip. The BLE Bluetooth communication chip has a small size, which can further reduce the design size of the product. The Bluetooth communication chip specifically uses the low-power BLE Bluetooth communication chip, which achieves a stable and convenient wireless data transmission function and can operate at low energy consumption and prolong the battery life of the device.

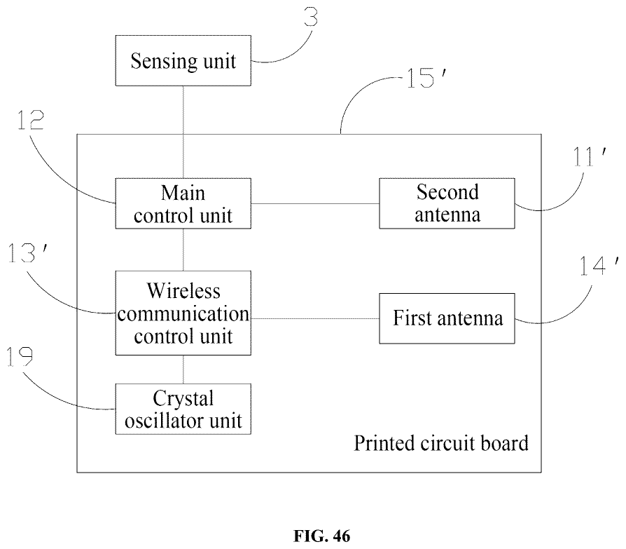

Referring to FIG. 46, FIG. 46 is a circuit diagram of the control module the detector shown in FIG. 36. The control module 1 further includes a crystal oscillator element 19. The crystal oscillator element 19 is electrically connected to the wireless communication control unit 13 and is configured to provide a basic clock for the wireless communication control unit 13. Specifically, in this embodiment, a crystal oscillation frequency of the crystal oscillator element 19 is 32 MHz. The crystal oscillator element 19 is crystal oscillator, which is an electronic element that uses a piezoelectric effect of a quartz crystal to generate stable frequency oscillation. The crystal oscillator element 19 plays a role of providing a clock signal in an electronic device and is a key component to ensure normal operation of the device.

The main control unit 12′ uses an AFE chip, which is configured to transmit detected data to the wireless communication control unit 13 through an SPI/I2C/UART or a GPIO interface. The AFE chip, namely an analog front end chip, is an electronic element that plays an important role in signal acquisition and processing; the serial peripheral interface (SPI), the inter-integrated circuit (I2C), the universal asynchronous receiver/transmitter (UART), and the general-purpose input/output (GPIO) are several commonly used interfaces and communication protocols in electronic systems. In this embodiment, the interface can use an SPI, an I2C interface, a UART interface, or a GPIO interface. The specific interface to be used in the product is selected according to a specific device communication requirement. This embodiment of the present disclosure does not impose any specific limitation. The SPI/I2C/UART or GPIO interface is used for data interaction with the main control unit 12′, thereby optimizing the transmission efficiency of the detected data and improving the overall performance and stability of a wireless communication system.

It is worth mentioning that through the proper layout and design of the second antenna 11′, the main control unit 12′, the wireless communication control unit 13, the first antenna 14′, the battery module 16, the shielding assembly 17′, the sensing unit connector 18, and other components on the detection circuit board 15′, the performance of the NFC antenna and the performance of the steel sheet antenna can be achieved on the detection circuit board 15′, thus achieving an optimized design in terms of the size and the costs.

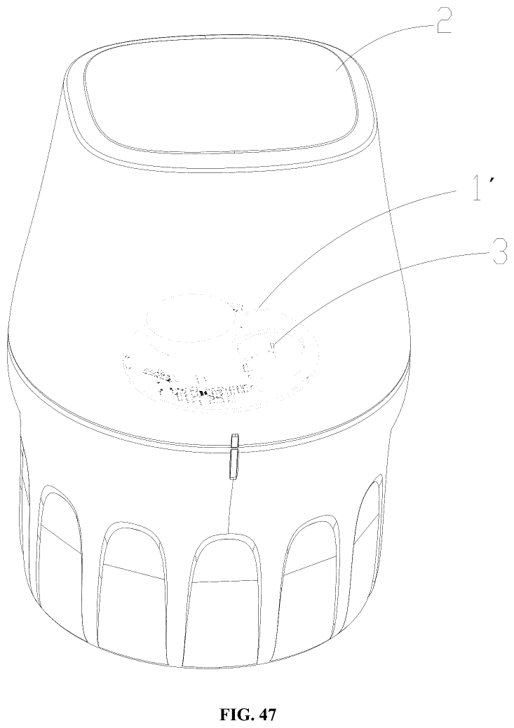

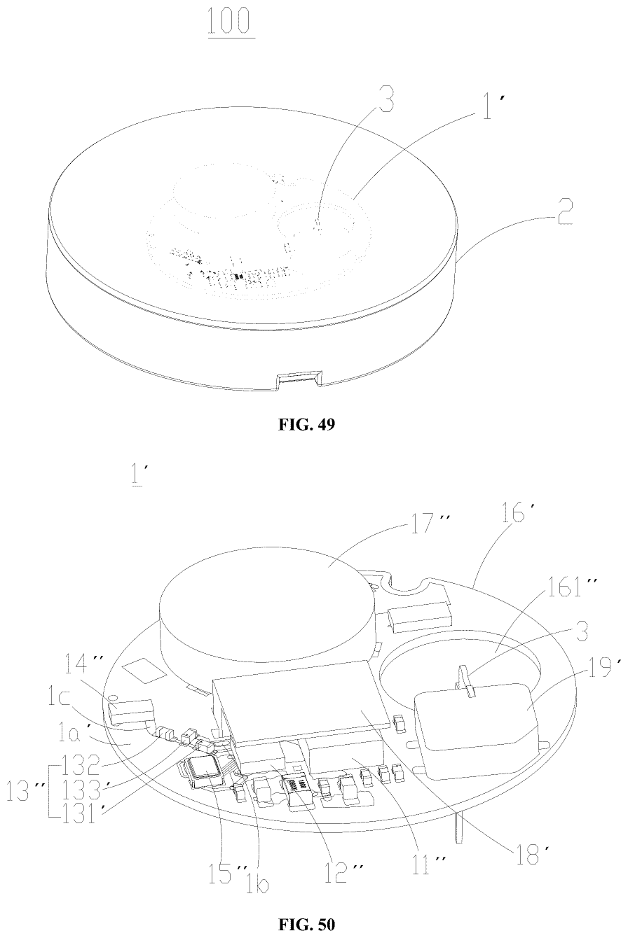

Referring to FIG. 47 and FIG. 49, FIG. 47 and FIG. 49 are schematic diagrams of partial structures of two shapes of a detector 100 according to an embodiment of the present disclosure. It can be understood that other parts (not shown) such as a guide needle, a bracket, an elastic element, a pressing element, and a sealing element are further arranged in the detector 100. Certainly, the detector 100 can be specifically designed according to a specific application scenario, and may not include the foregoing parts not shown. As the parts not shown in the accompanying drawings are not the key innovation technology points of the present disclosure, these parts will not be described in detail and will not be shown in the accompanying drawings in this embodiment.

The detector 100 is a blood glucose detector configured to measure a blood glucose level of a user or a patient. The detector 100 may also be a continuous detector, which is worn on a specific body part of a user or a patient, for continuous monitoring, data analysis, and management.

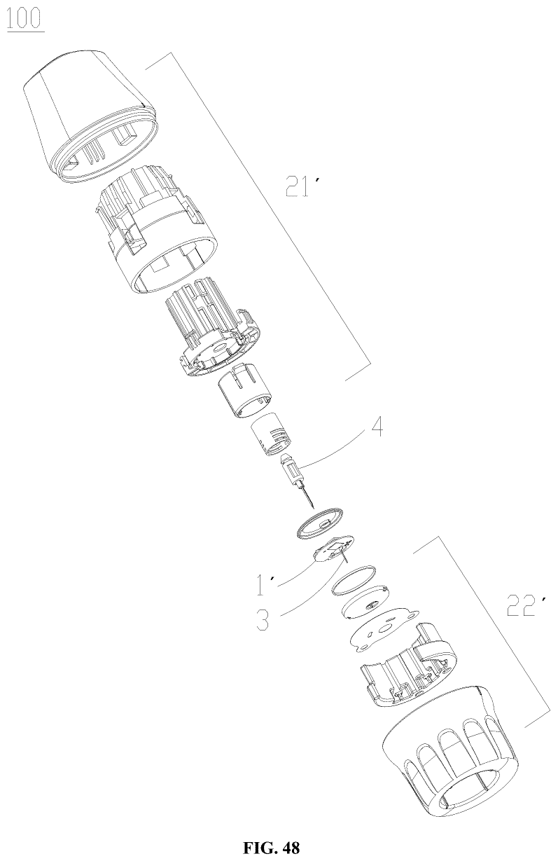

Specifically, referring to FIG. 48, FIG. 48 is a three-dimensional exploded view of the detector 100 shown in FIG. 47. In this embodiment, the detector 100 includes a housing 2, and an electric control module 1, a sensing unit 3, and a guide needle 4 which are arranged inside the housing 2. The guide needle 4 is configured to: pierce a sampling site to obtain a sample, and obtain raw data from the sample through the sensing unit 3 or transmit the sample to the electric control module 1 for processing, conversion, and transmission. In this embodiment, the housing 2 includes an upper housing assembly 21′ and a lower housing assembly 22′. The upper housing assembly 21′ and the lower housing assembly 22′ are enclosed to form a cavity. The electric control module 1, the sensing unit 3, and the guide needle 4 are all arranged inside the cavity. Since the housing 2 is not an innovative design point of this embodiment of the present disclosure, it will not be elaborated here.

Referring to FIG. 49, FIG. 49 is a schematic diagram of a partial structure of another detector 100 according to a fourteenth embodiment of the present disclosure. In this embodiment, the detector 100 may not include the probe. The detector 100 can be directly worn on a body part of the user or the patient to continuously monitor blood glucose of the user or the patient, and transmit monitored data to the electric control module 1 for processing, conversion, and transmission through the sensing unit 3. In this embodiment, the detector 100 includes a housing 2 and an electric control module 1 arranged inside the housing 2. The electric control module 1 processes, converts, and transmits acquired raw data. The detector may further include a sensing unit 3. The sensing unit 3 is configured to: obtain relevant raw data of the blood glucose and transmit the raw data to the electric control module 1 for processing, conversion, and transmission.

The electric control module 1′ in the foregoing embodiment may be connected to an external electronic device such as a mobile phone, a tablet, and a computer, and process, convert, and transmit the blood glucose detected data to external device, thereby facilitating subsequent big data analysis and organization, and facilitating blood glucose level management for the user. It can be understood that the sensing unit 3 may be equivalent to a probe the above implementation I to implementation XI.

Based on FIG. 47 and FIG. 48, it can be seen that the specific structure and appearance of the housing 2 of the detector 100 are designed according to a product design requirement. The structure of the housing 2 shown in FIG. 47 and FIG. 49 is only a schematic diagram for ease of understanding.

Due to different application scenarios and different internal structures of the detector 100, other parts are not the key innovative technology points of this embodiment of the present disclosure. Therefore, the following embodiments of the present disclosure will provide a detailed description for the key innovative part, namely, the electric control module 1′, in conjunction with the accompanying drawings.

Referring to FIG. 50, it is a three-dimensional diagram of an electric control module 1′ of a detector 100 according to an embodiment of the present disclosure. The electric control module 1′, as a part of the detector 100, includes a circuit board. The circuit board is specifically a detection circuit board 16′. The other components of the electric control module 1 are all arranged on the detection circuit board 16′. The detector 100 includes a sensing unit 3 configured to: obtain relevant raw data, acquired by the detector 100, of blood glucose of a user or a patient and transmit the raw data to the electric control module 1′ for processing. The detection circuit board 16′ has a first mounting site 161″ configured to arrange the sensing unit 3. The first mounting site 161″ is a hole that penetrates through the detection circuit board 16′.

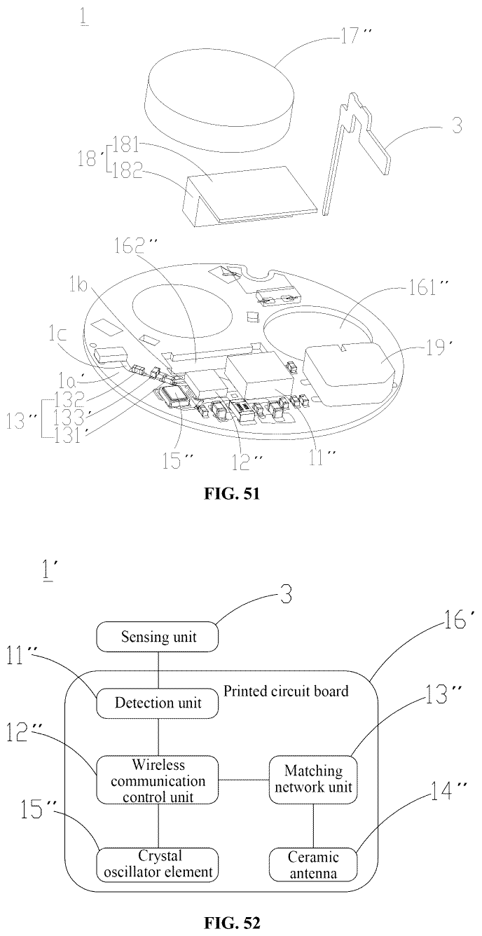

Specifically, referring to FIG. 51 and FIG. 52, the electric control module 1′ includes a detection unit 11″, a wireless communication control unit 12″, a matching network unit 13″, and a ceramic antenna 14″. The detection unit 11″, the wireless communication control unit 12″, the matching network unit 13″, and the ceramic antenna 14″ are all arranged on the detection circuit board 16′.

The detection unit 11″ is configured to process the raw data output by the sensing unit 3 to obtain detected data. The wireless communication control unit 12″ is electrically connected to the detection unit 11″, and is configured to: receive the detected data and output a radio frequency signal. The matching network unit 13′ is electrically connected to the wireless communication control unit 12″ to: receive the radio frequency signal and perform network matching. The ceramic antenna 14″ is electrically connected to the matching network unit 13′, and is configured to receive a network-matched radio frequency signal for wireless transmission when the matching network unit 13′ reaches a matched state.

Compared with the existing technology, the electric control module 1′ provided in the present disclosure processes and converts the detected data and transmits the detected to an external interactive device through connection and cooperation of the detection unit 11″, the wireless communication control unit 12″, the matching network unit 13″, and the ceramic antenna 14″, thus achieving efficient data processing, wireless communication, and data transmission, which facilitates analysis and management by using big data. In addition, the ceramic antenna 14″ with a small size is used, which further reduces the overall size of the detector 100, thereby miniaturizing the product.

Specifically, the ceramic antenna 14″ is a patch ceramic antenna, specifically ANT016008LCS2442MA2. A package size of the ceramic antenna 14″ may be 1608 or 0603. 1608 or 0603 means a size code of an electronic element, which is a size of a standardized electronic element. Specifically, “16” indicates that a length of an element is 1.6 millimeters; “08” indicates that a width of an element is 0.8 millimeters; ‘06’ indicates that a length of an element is 0.6 millimeters; and ‘03’ indicates that a width of an element is 0.3 millimeters. The ceramic antenna 14″ is packaged in 1608, so that the ceramic antenna 14″ can be seamlessly integrated into a compact design of the device and maintain good wireless communication performance. In addition, a standard size is used, so that it is suitable for assembling of an automated surface mount technology (SMT), which can further reduce the production cost of the product. It can be understood that the ceramic antenna 14″ has a smaller size because of the package size of 0603, which has the technical effect of better portability.

The wireless communication control unit 12″ includes a Bluetooth communication chip. The Bluetooth communication chip is a BLE Bluetooth communication chip. The BLE Bluetooth communication chip has a small size, which can further reduce the design size of the product. The Bluetooth communication chip specifically uses the low-power BLE Bluetooth communication chip, which achieves a stable and convenient wireless data transmission function and can operate at low energy consumption and prolong the battery life of the device.

In this embodiment, an antenna clearance zone 1a′ is reserved around the ceramic antenna 14″. The antenna clearance zone 1a′ is a space between an antenna and its surrounding objects, which is crucial for the performance of the antenna. The antenna clearance zone 1a′ ensures that the antenna has a sufficient space to avoid shielding or interference, thus ensuring an omnidirectional communication effect of the ceramic antenna 14″. Arranging the antenna clearance zone 1a′ around the ceramic antenna 14″ can ensure that a signal of the antenna is less obstructed, thereby improving the transmission quality and coverage range of the signal of the antenna.

A distance between the ceramic antenna 14″ and a copper pavement region on the detection circuit board 16′ where the ceramic antenna 14″ is located is greater than or equal to 0.70 mm. A distance between the ceramic antenna 14″ and an outer edge of the detection circuit board 16′ is greater than or equal to 1.0 mm. A distance between the ceramic antenna 14″ and other element regions on the detection circuit board 16′ is greater than or equal to 0.3 mm. The copper pavement region on the detection circuit board 16′ is also referred to as a copper-clad region or a ground layer. The distance limitation on the copper pavement region on the detection circuit board 16′ where the ceramic antenna 14″ is located can avoid the impact on the signal emission performance. Meanwhile, sufficient distances (at least 0.3 mm and 1.0 mm) is maintained between the ceramic antenna 14″ and the outer edge of the detection circuit board 16′, as well as other element regions, thereby optimizing the layout of the circuit board and the signal transmission, avoiding potential interference, improving the stability and overall electromagnetic compatibility of the antenna, and ensuring the performance and safety of a radio communication device.

Further, the electric control module 1′ further includes a crystal oscillator element 15″. The crystal oscillator element 15″ is electrically connected to the wireless communication control unit 12″ and is configured to provide a basic clock for the wireless communication control unit 12″. Specifically, in this embodiment, a crystal oscillation frequency of the crystal oscillator element 15″ is 32 MHz. The crystal oscillator element 15″ is crystal oscillator, which is an electronic element that uses a piezoelectric effect of a quartz crystal to generate stable frequency oscillation. The crystal oscillator element 15″ plays a role of providing a clock signal in an electronic device and is a key component to ensure normal operation of the device.

The detection unit 11″ is configured to transmit the detected data to the wireless communication control unit 12″ through an SPI/I2C/UART or GPIO interface. The serial peripheral interface (SPI), the inter-integrated circuit (I2C), the universal asynchronous receiver/transmitter (UART), and the general-purpose input/output (GPIO) are several commonly used interfaces and communication protocols in electronic systems. In this embodiment, the interface can use an SPI, an I2C interface, a UART interface, or a GPIO interface. The specific interface to be used in the product is selected according to a specific device communication requirement. This embodiment of the present disclosure does not impose any specific limitation. The SPI/I2C/UART or GPIO interface is used for data interaction with the detection unit 11″, thereby optimizing the transmission efficiency of the detected data and improving the overall performance and stability of a wireless communication system.

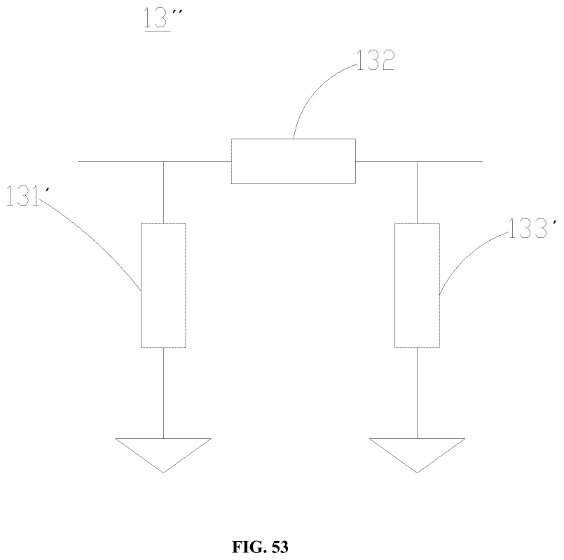

Referring to FIG. 53, FIG. 53 is a circuit diagram of a matching network unit 13′ in an electric control module 1′ according to an embodiment of the present disclosure. Specifically, the matching network unit 13′ is a x-type network. Specifically, the matching network unit 13′ includes a first matching unit 131′, a second matching unit 132, and a third matching unit 133′. Two ends of the second matching unit 132 are respectively connected to the wireless communication control unit 12″ and the ceramic antenna 14″. The first matching unit 131′ is connected between a node between the wireless communication control unit 12″ and the ceramic antenna 14″, and a ground. The third matching unit 133′ is connected between a node between the wireless communication control unit 12″ and the second matching unit 132, and the ground. The first matching unit 131′ is an inductive or capacitive element. The second matching unit 132 is an inductive or capacitive element. The third matching unit 133′ is an inductive or capacitive element. It can be understood that any one, two, or all of the first matching unit 131′, the second matching unit 132, and the third matching unit 133′ may be inductive or capacitive elements. The present disclosure does not impose a limitation. Specifically, in this embodiment, the first matching unit 131′ is not welded. The second matching unit 132 is a capacitive element, specifically 33 pF. The third matching unit 133′ is an inductive element, specifically 2.2 nH. By arranging the π-type network between the wireless communication control unit 12″ and the ceramic antenna 14″, a good transmission effect and transmission stability of a wireless communication signal can be achieved, thus improving the signal receiving and transmitting efficiency, improving the reliability of wireless communication, effectively reducing the energy consumption, and reducing the impact on other radio devices.

Specifically, the matching network unit 13′ and the wireless communication control unit 12″ are electrically connected through a first microstrip 1b formed on the detection circuit board 16′, and the matching network unit 13′ and the ceramic antenna 14″ are electrically connected through a second microstrip 1c formed on the detection circuit board 16′. The microstrip is a commonly used transmission line form in microwave and millimeter wave frequency bands. Its characteristic is that an electromagnetic field is mainly limited to a thin dielectric layer, and transmission and control of electromagnetic waves are achieved through three basic parts: a patch, a ground plate, and a dielectric substrate. In this embodiment, a characteristic impedance of the first microstrip 1b and a characteristic impedance of the second microstrip 1c are 50 Ω. By implementing electrical connection through the microstrips, the layout and wiring between the units are simplified, so that the stability of the circuit is improved, the electromagnetic interference is reduced; frequency matching is optimized; and the performance of a wireless communication devices is improved.

To achieve power supplying, the electric control module 1′ further includes a battery module 17″. The battery module 17″ is arranged on the detection circuit board 16′.

The detector 100 provided in this embodiment of the present disclosure needs to be irradiated for sterilization when configured to monitor blood glucose. To protect a sensitive component in the electric control module 1′ from being damaged, the electric control module 1′ further includes a shielding assembly 18′. The shielding assembly 18′ is arranged on one side of the detection unit 11″ to block at least part of an irradiation ray, thus forming a full shadow shielding region to protect the detection unit 11″. The detection circuit board 16′ has a second mounting site 162. The second mounting site 162 is configured to arrange the shielding assembly 18′. The shielding assembly 18′ includes a shielding block 181 and/or a shielding bracket 182. The second mounting site 162 is a hole that penetrates through the detection circuit board 16′. Specifically, in this embodiment of the present disclosure, the shielding assembly 18′ includes a shielding block 181 and a shielding bracket 182. The shielding bracket 182 is arranged at the second mounting site 162 and supports the shielding block 181. The shielding block 181 is placed above the detection unit 11″ to form the full shadow shielding region that blocks the irradiation ray in a direction. A density of the shielding block 181 is greater than 1000 kilograms per cubic meter. A larger density of the shielding block 181 indicates that the shielding block 181 has a better effect of blocking the irradiation ray. The shielding block 181 may be plate-like or block-shaped or in other shapes. This embodiment does not impose a specific limitation. In other embodiments, the shielding assembly 18′ may only include the shielding block 181, as long as it can block the irradiation ray and form the full shadow shielding region. In another embodiment, the full shadow shielding region may be formed through the shielding bracket 182.

Specifically, the battery module 17″, the shielding assembly 18″, and the detection unit 11″ are located on one straight line, and the battery module 17″ and the shielding assembly 18″ are configured to jointly block at least part of the irradiation ray, thereby ensuring that the irradiation ray in this direction is blocked. Certainly, in other embodiments, only the battery module 17″ can be used to shield and block the irradiation ray.

The wireless communication control unit 12″ is arranged in the full shadow shielding region. The electric control module 1′ further includes a sensing unit connector 19′. The sensing unit connector 19′ is arranged on the detection circuit board 16′ for electrical connection between the sensing unit 3 and the detection unit 11″. Arranging the full shadow shielding region can effectively prevent damage to the detection unit 11″ and the wireless communication control unit 12″ caused by the irradiation ray, thereby ensuring the detection accuracy and service life of the electric control module 1′.

Specifically, to further reduce the volume of the electric control module 1 to achieve the miniaturization of the detector 100, in this embodiment, the detection circuit board 16′ is circular, and the battery module 17″, the first mounting site 161″, the sensing unit connector 19′, the detection unit 11′, the wireless communication control unit 12″, the matching network unit 13′, and the ceramic antenna 14″ are arranged in sequence around the second mounting site 162. This design can make the electric control module 1 have a more compact layout design and a smaller volume.

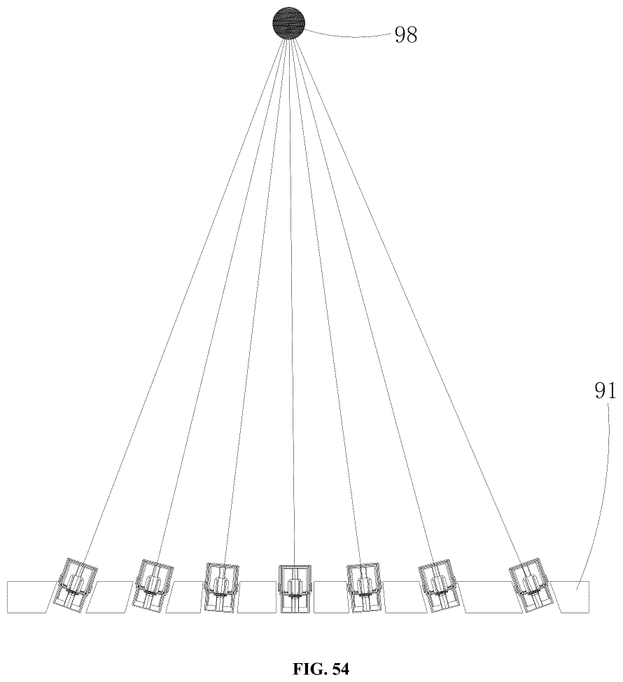

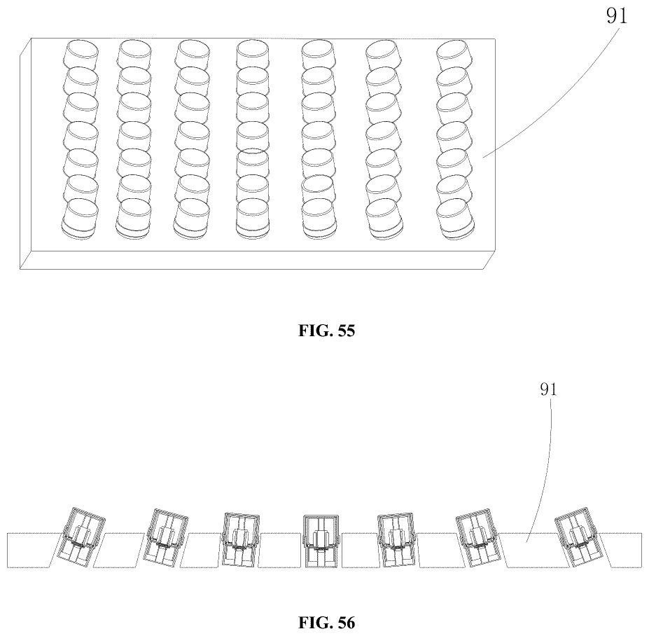

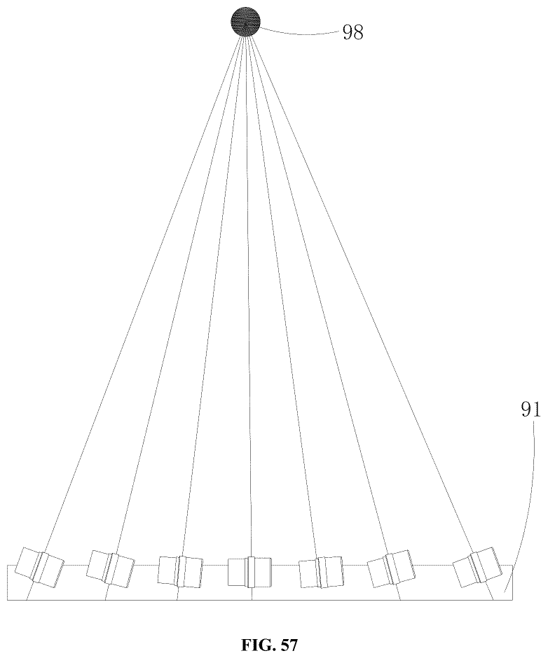

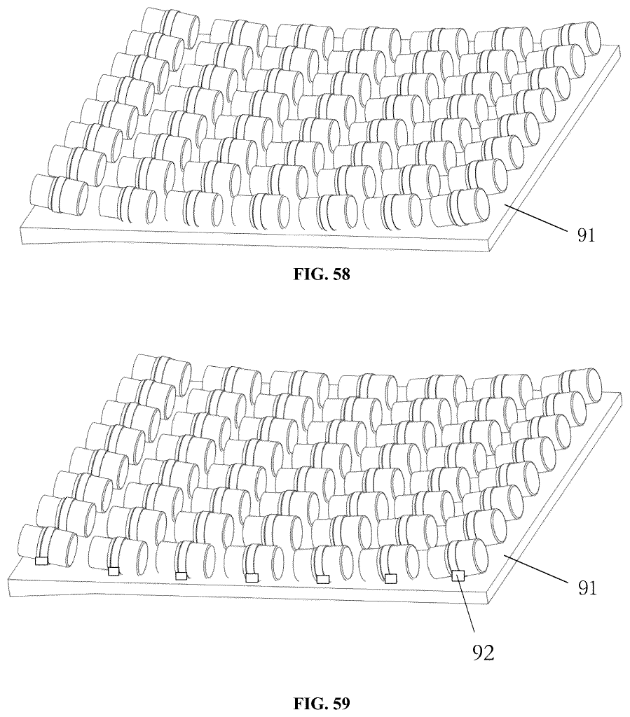

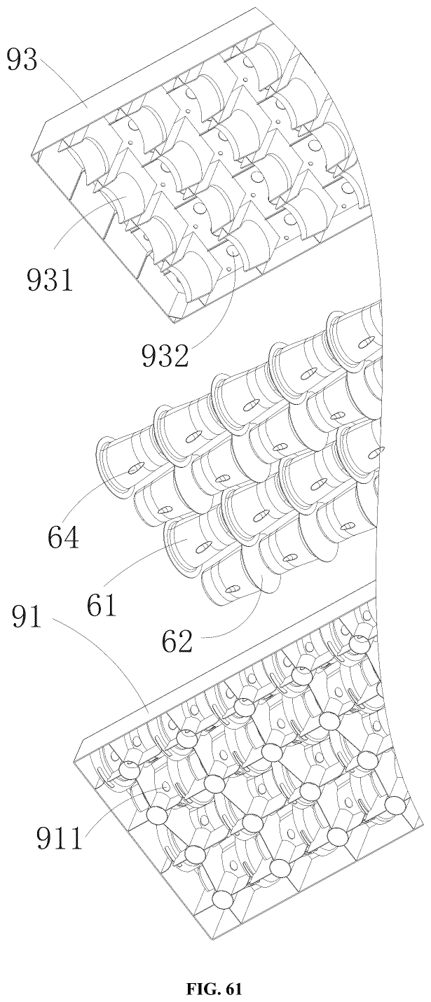

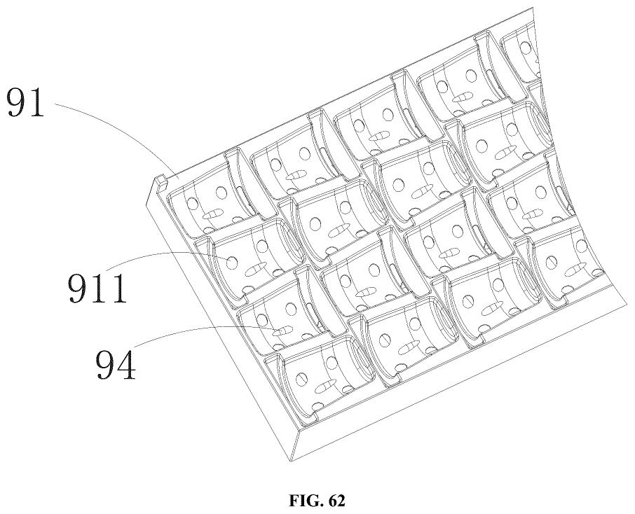

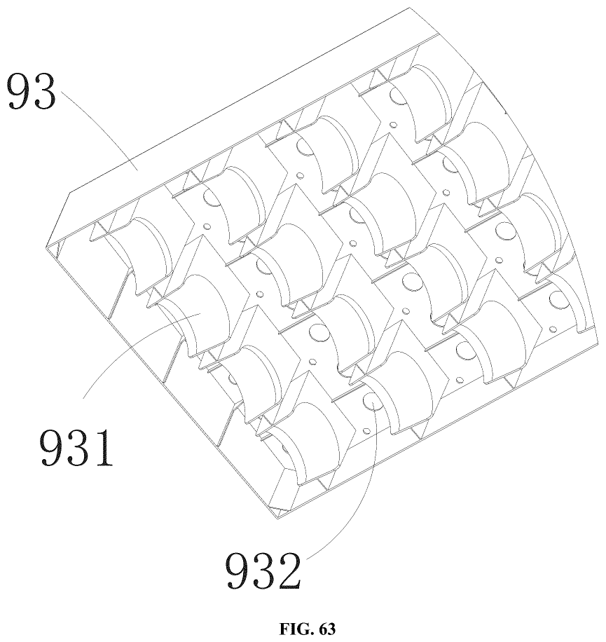

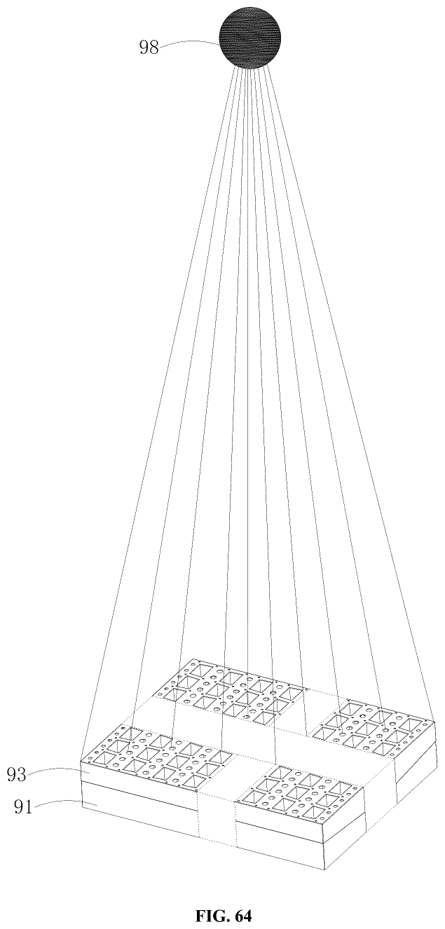

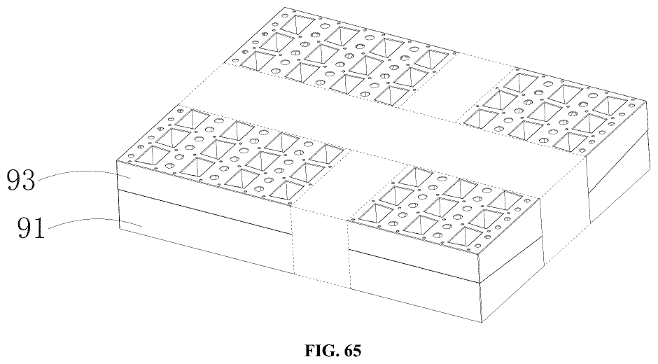

Please combine with FIGS. 1-53 and refer to FIGS. 54-65, embodiments of the present disclosure further provide a sterilization system, and the sterilization system includes a bearing frame 91, and the bearing frame 91 is used for cooperating with the irradiation source 98 for sterilization; and
the bearing frame 91 is located on one side of the irradiation source 98, one side of the bearing frame 91 that faces the irradiation source 98 is provided with a plurality of bearing portions, and each bearing portion can place one detector of any embodiment above.

When the detector is placed on the bearing portions, the bearing portions are used for correcting the orientation of the detector, such that the shielding assembly in the detector is located on the path that the irradiation source 98 is emitted to the detector.

The irradiation source 98 can emit various types of irradiation rays, including but being not limited to an X-ray, an electronic beam and a gamma ray.

When a plurality of detectors are subjected to irradiation for sterilization at once, the plurality of detectors may be placed in different bearing portions of the bearing frame 91. Since the rays emitted from the irradiation source 98 are in multiple directions, to ensure that each detector can be exposed to the irradiation ray, the bearing frame 91 is used for correcting the orientation of the detector located therein in this embodiment, to ensure that the shielding assembly or the shielding block 80 can implement the shielding effect correctly, that is, the total-shadow shielding zone 801 that wraps the sensitive element 221 and is not involved by the irradiation ray is formed.

Further, in this embodiment, a center of the bearing frame 91 is set correspondingly to the irradiation source 98;
the bearing portions may be bearing slots disposed in the bearing frame 91, the bearing slots in the middle of the bearing frame 91 are disposed perpendicularly, and other bearing slots adjacent to the middle bearing slots tilt towards one side of the irradiation source 98; and
the farther the bearing slots are from the irradiation source 98, the greater a tilt angle is.

Specifically, the bearing slots in the middle of the bearing frame 91 directly face the irradiation source 98, an included angle formed by the irradiation ray emitted from the irradiation source 98 and the surface of the bearing frame 91 is a right angle, so the bearing slots are perpendicularly disposed. The farther from the irradiation source 98, the smaller the included angle formed by the irradiation ray emitted from the irradiation source 98 and the surface of the bearing frame 91 is, relatively adjusting the tilt angles of the bearing slots enables the central axes of all bearing slots can be all emitted to the irradiation source 98, to ensure that the shielding assembly or the shielding block 80 can implement the shielding effect correctly, that is, the total-shadow shielding zone 801 that wraps the sensitive element 221 and is not involved by the irradiation ray is formed.

Specifically, the placement mode of the detector is adjusted according to the forming mode and position of the total-shadow shielding zone, and the detector can be placed in the bearing frame 91 in a basically perpendicular way or placed in the bearing frame 91 in a basically horizontal way.

Further, please refer to FIG. 59, in yet another embodiment, the sterilization system may also include at least one fastener 92 of a magnetic suction fastener and a clip fastener, and the fastener 92 is used for fixing the detector on the bearing frame.

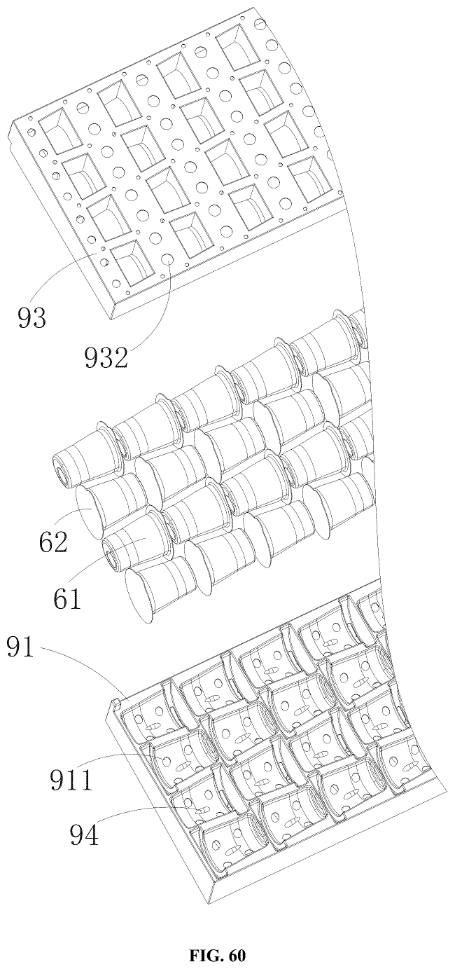

Please combine with FIGS. 60-65, embodiments of the present disclosure further provide a sterilization system, the sterilization system has the structure that is basically the same as that of the sterilization system in the above embodiments, where the components with the structures that are basically the same adopt the same number, the above components are not repeatedly described any more, and the key part of the sterilization system provided by this embodiment and the part different from other embodiments are mainly described below.

In this embodiment, the bearing portions are also provided with a plurality of first cooling holes 911; the sterilization system further includes a cover plate 93, and the cover plate 93 is used for covering one side where the detector is located to fix the detector in cooperation with the bearing portions; and the cover plate 93 has a counterpoint slot 931 for accommodating at least part of the detector, and a plurality of second cooling holes 932.

Further, the main housing 61 of the outer package may also be provided with a third counterpoint structure 64. The third counterpoint structure 64 is used for counterpoint fit with a fourth counterpoint structure 94 on the bearing frame 91 of the sterilization system, such that the orientation of the detector may be fixed, facilitating the sterilization for the detector by using the irradiation ray in the preset direction. In this embodiment, the third counterpoint structure 64 is a counterpoint groove, and the fourth counterpoint structure 94 is a counterpoint bulge. It can be understood that, as shown in FIG. 32, in another alternative embodiment, when the detector is not provided with the outer package, the third counterpoint structure 64 may be disposed on the outer surface (such as the outermost housing) of the detector.

Specifically, in this embodiment, the main housing 61 is also provided with a first counterpoint structure 63, the bottom (such as on the second outer shell 52) of the detector is provided with a second counterpoint structure 56, and the second counterpoint structure 56 is in counterpoint fit with the first counterpoint structure 63, such that the detector can be disposed in the main housing 61 according to the preset orientation. Such setting can ensure that the sensitive element 221 can be located in the scope of the total-shadow shielding zone 801 when the irradiation ray irradiates the detector for sterilization, thus achieving the protection to the sensitive element 221.

Other compositions for the detector and sterilization system in the above embodiments can adopt various technical solutions that are known by those skilled in the art now or in the future, and detailed descriptions are not made here.

In the description of the present disclosure, It is to be understood that, The terms “center”, “longitudinal”, “transverse”, “upper”, “lower”, “front”, “rear”, “left”, “right”, “vertical”, “horizontal”, “top”, “bottom”, “inner”, “outer”, “clockwise”, “counterclockwise”, and the like indicate azimuth or positional relationships based on the azimuth or positional relationships shown in the drawings, For purposes of convenience only of describing the present disclosure and simplifying the description, Rather than indicating or implying that the indicated device or element must have a particular orientation, be constructed and operated in a particular orientation, therefore, not to be construed as limiting the present disclosure.

In addition, The terms “first” and “second” are used for descriptive purposes only, While not to be construed as indicating or implying relative importance or implicitly specifying the number of technical features indicated thereby, features defining “first,” “second,” and “second” may explicitly or implicitly include one or more of the described features. In the description of the present disclosure, “multiple” means two or more unless explicitly specified otherwise.

In the description of the present disclosure, it is to be noted that unless otherwise expressly specified and defined, the terms “mounted”, “connected”, and “connected” are to be construed broadly, for example, as either a fixed connection, or a detachable connection, or an integral connection, either a mechanical connection, or an electrical connection. The specific meaning of the above term in the present disclosure will be understood by those of ordinary skill in the art depending on the particular circumstances, either directly or indirectly via an intermediate medium, communication between the two elements, or interaction between the two elements. The specific meanings of these terms in the present disclosure will be understood by those of ordinary skill in the art as the case may be.

In the present disclosure, unless specific regulation and limitation otherwise, the first feature “onto” or “under” the second feature may include the direct contact of the first feature and the second feature, or may include the contact of the first feature and the second feature through other features between them instead of direct contact. Moreover, the first feature “onto”, “above” and “on” the second feature includes that the first feature is right above and obliquely above the second feature, or merely indicates that the horizontal height of the first feature is higher than the second feature. The first feature “under”, “below” and “down” the second feature includes that the first feature is right above and obliquely above the second feature, or merely indicates that the horizontal height of the first feature is less than the second feature.

Many different implementation modes or examples are provided in the above disclosure, so as to implement different structures of the present disclosure. To simplify the disclosure of the present disclosure, the components and setting of specific examples are described above. Of course, they are merely exemplary, and not intended to limit the present disclosure. In addition, the reference figures and/or reference letters may be repeated in different examples of the present disclosure, and this repetition is for the purpose of simplification and clarity, and does not indicate the discussion for the relation between various implementation modes and/or settings by itself.

In conclusion, the above are only the specific implementation modes of the present disclosure, but the scope of protection of the present disclosure is not limited to this. Those skilled in the art can easily think of various changes or replacements within the scope of the technology disclosed in the present disclosure, which shall be covered by the scope of protection of the present disclosure. Therefore, the scope of protection of this application should be subject to the scope of protection of the appended claims.

What is claimed is:

1. A detector, comprising a housing assembly, a detection assembly, and a shielding assembly, wherein
the detection assembly comprises a first housing, a detection circuit board and a probe, wherein the detection circuit board is arranged in the first housing and is electrically connected with the probe; the detection circuit board comprises a sensitive element; a first end of the probe is fixed in the first housing, and a second end extends out of the first housing;
the housing assembly comprises a collision housing and a pressing portion that are capable of sliding relative to each other; the detection assembly is located below a bottom of the pressing portion; the collision housing is used for abutting against a sampling part;
the pressing portion is used for driving the detection assembly to move towards the sampling part, so as to pierce the probe into the sampling part for detection;
when the detector is sterilized through an irradiation ray, the shielding assembly is used for blocking part of the irradiation ray to protect the sensitive element; the shielding assembly comprises a side plate connected to the detection circuit board, and a top plate connected to the side plate; the side plate is adjacent to the sensitive element; and the top plate is located on one side of the sensitive element away from the detection circuit board.

2. The detector according to claim 1, wherein the first housing comprises a bearing shell and a cover body; the bearing shell is hermetically connected with the cover body and forms a sealed storage cavity; the detection circuit board is located in the storage cavity; the first housing further comprises a sealing element; the bearing shell is hermetically connected with the cover body through the sealing element; and the sealing element comprises a sealant, a sealing rubber ring or a combination of the sealant and the sealing rubber ring.

3. The detector according to claim 1, wherein the shielding assembly and the sensitive element are arranged on a path that an irradiation source for sterilization irradiates the detector, so that the shielding assembly blocks a radiation of the irradiation source to the sensitive element; and an irradiation direction of the radiation is different from a pressing direction of the pressing portion.

4. The detector according to claim 1, wherein the sensitive element comprises a main control unit arranged on the detection circuit board; the main control unit is adjacent to the side plate and is located between the top plate and the detection circuit board; the probe is electrically connected to the main control unit; and the main control unit processes original data of a detected object sampled by the probe to obtain detected data.

5. The detector according to claim 4, wherein the detection circuit board has a first mounting site for arranging the probe, and a second mounting site for arranging the shielding assembly; the side plate is arranged at the second mounting site in a penetrating manner; the shielding assembly further comprises a base plate; the base plate is connected to one end of the side plate away from the top plate; the base plate is located on one side of the detection circuit board away from the top plate; the top plate is provided with a connecting portion; the connecting portion is connected to one end of the side plate away from the base plate; the base plate is integrally formed with the side plate;
and the connecting portion is connected with the side plate in a welded, crimped, or adhered manner.

6. The detector according to claim 5, further comprising a probe connector, wherein the probe connector is arranged on the detection circuit board and is used for being electrically connected to the probe and the main control unit; the first mounting site comprises a head mounting region for mounting part of the probe in a penetrating manner, and a tail mounting region communicated to the head mounting region; the probe connector is arranged in the tail mounting region; each of the first mounting site and the second mounting site comprises an opening penetrating through the detection circuit board; the second mounting site has a length greater than or equal to 3 mm and a width greater than or equal to 1 mm; a diameter of the head mounting region is greater than 2.5 mm; and the tail mounting region has a length greater than 2.5 mm and a width greater than 1 mm.

7. The detector according to claim 4, further comprising a wireless communication control unit and a first antenna electrically connected to the wireless communication control unit, wherein the wireless communication control unit is electrically connected to the main control unit; the wireless communication control unit is configured to: receive the detected data and output a detection signal to the first antenna, so that the first antenna receives the detection signal for wireless transmission; and the wireless communication control unit is adjacent to the side plate and is located between the top plate and the detection circuit board.

8. The detector according to claim 7, wherein the detection circuit board further comprises a battery module or an electronic device; the battery module or the electronic device, the shielding assembly, and the sensitive element are all arranged on the path that the irradiation source for sterilization irradiates the detector, so that the battery module or the electronic device and the shielding assembly block the radiation of the irradiation source to the sensitive element together; and the side plate is located between the battery module and the sensitive element or the electronic device and the sensitive element.

9. The detector according to claim 8, wherein the battery module comprises a battery and a battery holder; the battery holder is connected to the detection circuit board; the battery is arranged between the battery holder and the detection circuit board; the battery holder comprises a main body portion and at least two welding legs, at least one blocking leg, and at least one push spring piece which are arranged on the main body portion; the main body portion is arranged on one side of the battery away from the detection circuit board; the at least two welding legs are opposite to each other and are connected to the detection circuit board; the blocking leg collides with the battery to limit the battery between the battery holder and the detection circuit board; the push spring piece collides with the battery to press the battery towards the detection circuit board; a height of the battery holder is greater than 1.5 mm; and a thickness of the main body portion is greater than 0.1 mm.

10. The detector according to claim 8, wherein the detection circuit board is circular;
the battery module, the first mounting site, the probe connector, the main control unit, and the wireless communication control unit are arranged in sequence around the second mounting site; and the first antenna is arranged around the battery module, the second mounting site, the wireless communication control unit, the main control unit, and the probe connector.

11. The detector according to claim 8, further comprising a second antenna, wherein the second antenna is arranged on the detection circuit board and is electrically connected to the main control unit and used for receiving an external wireless radio frequency signal, so that the main control unit controls the detector to start working according to the wireless radio frequency signal; and the second antenna is a near field communication (NFC) radio frequency antenna; and the wireless radio frequency signal is an NFC radio frequency signal.

12. The detector according to claim 11, wherein the second antenna comprises a conductive line formed on the detection circuit board; the second antenna is formed on a bottom layer and a sub-bottom layer of the detection circuit board through a winding layout; a number of turns of the second antenna is greater than or equal to four; the first antenna is arranged around a periphery of the second antenna; two ends of the second antenna are respectively connected to the main control unit and form a surrounding region; and the battery module, the first mounting site, the probe connector, the main control unit, the wireless communication control unit, and the second mounting site are all located in the surrounding region.

13. The detector according to claim 7, wherein the first antenna is a steel sheet antenna comprising an antenna main body arranged above the detection circuit board, and at least one feed portion connected to the antenna main body and the detection circuit board; the at least one feed portion is electrically connected to the wireless communication control unit through a microstrip; and the at least one feed portion is provided with an antenna clearance zone.

14. The detector according to claim 13, wherein the microstrip comprises a line formed on the detection circuit board; the at least one feed portion comprises a first pin, a second pin, a third pin, and a fourth pin; the first pin and the second pin are arranged at one end of the antenna main body; the fourth pin is arranged in the middle of the antenna main body; the third pin is arranged between the first pin or the second pin and the fourth pin; the first pin and the second pin are both feed ground pins; the third pin is an antenna feed point; the fourth pin is suspended or is a feed ground pin; the first pin and the second pin are close to the battery module; the third pin is close to the wireless communication control unit and is electrically connected to the wireless communication control unit through the microstrip; the fourth pin is close to the main control unit; the antenna clearance zone comprises a first clearance zone and a second clearance zone; the third pin is arranged in the first clearance zone; the fourth pin is arranged in the second clearance zone; and the first pin, the second pin, the third pin, and the fourth pin have the same heights.

15. The detector according to claim 14, wherein a height of the antenna main body is greater than 1 mm; a thickness of the antenna main body is greater than 0.1 mm; a width of the antenna main body is greater than 0.8 mm; a distance between a bonding pad of each of the third pin and the fourth pin and a copper pavement region on the detection circuit board is greater than 0.8 mm; a length of one end of the third pin away from the first pin and a length of one end of the antenna main body away from the first pin are greater than 15 mm; the detector further comprises a matching network; the matching network is electrically connected to the wireless communication control unit and the steel sheet antenna via the microstrip; the matching network is a T-shaped network; the matching network comprises a first matching unit, a second matching unit, and a third matching unit; one of the first matching unit and the second matching unit is connected to the wireless communication control unit, and the other one of the first matching unit and the second matching unit is connected to the steel sheet antenna; the third matching unit is connected between a node between the first matching unit and the second matching unit, and a ground; the first matching unit is an inductive or capacitive element; the second matching unit is an inductive or capacitive element; and the third matching unit is an inductive or capacitive element.

16. The detector according to claim 7, wherein the first antenna is a ceramic antenna, which is electrically connected to the matching network and is configured to receive a network-matched radio frequency signal for wireless transmission when the matching network reaches a matched state.

17. The detector according to claim 16, wherein the ceramic antenna is a patch ceramic antenna; a package size of the ceramic antenna is 1608; the wireless communication control unit comprises a Bluetooth communication chip; the Bluetooth communication chip is a Bluetooth low energy (BLE) Bluetooth communication chip; an antenna clearance zone is reserved around the ceramic antenna; a distance between the ceramic antenna and the copper pavement region on the detection circuit board where the ceramic antenna is located is greater than or equal to 0.70 mm; a distance between the ceramic antenna and an outer edge of the detection circuit board is greater than or equal to 1.0 mm; and a distance between the ceramic antenna and another element region on the detection circuit board is greater than or equal to 0.3 mm.

18. The detector according to claim 7, further comprising a temperature sensor, wherein the temperature sensor is electrically connected to the main control unit or the wireless communication control unit and is configured to: acquire temperature information and transmit the information to the main control unit or the wireless communication control unit; the wireless communication control unit comprises a Bluetooth communication chip; the Bluetooth communication chip is a BLE Bluetooth communication chip; the control module further comprises a crystal oscillator element;
the crystal oscillator element is electrically connected to the wireless communication control unit and is configured to provide a basic clock for the wireless communication control unit; the main control unit is configured to transmit the detected data to the wireless communication control unit through a serial peripheral interface (SPI)/inter-integrated circuit (I2C)/universal asynchronous receiver/transmitter (UART) or a general-purpose input/output (GPIO) interface.

19. A sterilization system, comprising a bearing frame, wherein the bearing frame configured to cooperate with an irradiation source for sterilization;
the bearing frame is located on one side of the irradiation source; one side of the bearing frame that faces the irradiation source is provided with a plurality of bearing portions; each bearing portion is configured to place a detector; the detector comprises a housing assembly, a detection assembly, and a shielding assembly; and
when the detector is placed on each bearing portion, the bearing portion is configured to correct an orientation of the detector, so that the shielding assembly in the detector is located on a path that the irradiation source irradiates the detector.

20. The sterilization system according to claim 19, wherein a center of the bearing frame is arranged correspondingly to the irradiation source;
the bearing portions are bearing slots provided in the bearing frame; the bearing slot in the middle of the bearing frame is vertical, and other bearing slots adjacent to the middle bearing slot tilt towards one side of the irradiation source;
and if the bearing slots are farther from the irradiation source, tilting angles of the bearing slots are larger;
the sterilization system further comprises at least one fastener of a magnetic suction fastener and a clip fastener; the fastener is configured to fix the detector on the bearing frame; the bearing portions are further provided with a plurality of first cooling holes; the sterilization system further comprises a cover plate; the cover plate is configured to cover one side where the detector is located to fix the detector in cooperation with the bearing portions; the cover plate has a counterpoint slot for accommodating at least part of the detector, and a plurality of second cooling holes;
the bearing portions are further provided with counterpoint structures that are used in cooperation with the detector or another counterpoint structure on an outer package of the detector, so that the detector is capable of being arranged according to a preset orientation; and when the outer package of the detector has another counterpoint structure, the detector has a first counterpoint structure, and the outer package has a second counterpoint structure used in cooperation with the first counterpoint structure, so that the detector is arranged in the outer package according to the preset orientation.
